SEQUENCE LISTING (79 sequences)

SEQ ID NO: 1 - protein, 6 aa, synthetic construct
  SITE 1: note = Acetylated residue
KGDEVD

SEQ ID NO: 2 - protein, 6 aa, synthetic construct
  SITE 1: note = Maleimide modified residue
  SITE 6: note = p-aminobenzylcarbamate-Exatecan modified residue
KGDEVD

SEQ ID NO: 3 - protein, 4 aa, synthetic construct
  SITE 1: note = Maleimide modified residue
  SITE 4: note = p-aminobenzylcarbamate-monomethyl auristatin E modified residue
DEVD

SEQ ID NO: 4 - protein, 4 aa, synthetic construct
DEVD

SEQ ID NO: 5 - protein, 4 aa, synthetic construct
DLVD

SEQ ID NO: 6 - protein, 4 aa, synthetic construct
DEID

SEQ ID NO: 7 - protein, 4 aa, synthetic construct
LEHD

SEQ ID NO: 8 - protein, 6 aa, synthetic construct
KGDEVD

SEQ ID NO: 9 - protein, 450 aa, synthetic construct
EVQLVESGGGLVQPGGSLRLSCAASGFTFSSYAMSWVRQAPGKGLEWVSAISSDGGSTYYADSVKGRFTISRDNSKNTLYLQMNSLRAEDTAVYYCAKRRLWSYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 450 aa, synthetic construct
EVQLVESGGGLVQPGGSLRLSCAASGFTFSDYAMSWVRQAPGKGLEWVSAISSSGGSTYYADSVKGRFTISRDNSKNTLYLQMNSLRAEDTAVYYCAKLYYTYEVLDIWGQGTFVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 451 aa, synthetic construct
QVQLVQSGAEVKKPGSSVKVSCKASGGTFSSYYMHWVRQAPGQGLEWMGRISPGGGGTIYAQKFQGRVTITADESTSTAYMELSSLRSEDTAVYYCARAQSEGLSYYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 453 aa, synthetic construct
EVQLVESGGGLVQPGGSLRLSCAASGFTFSDYAMSWVRQAPGKGLEWVSRISSSGSSKYYADSVKGRFTISRDNSKNTLYLQMNSLRAEDTAVYYCAKWGSYGYGLVYYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 448 aa, synthetic construct
EVQLVESGGGLVQPGGSLRLSCAASGFTFSDYAMSWVRQAPGKGLEWVSRISGSGGTKYYADSVKGRFTISRDNSKNTLYLQMNSLRAEDTAVYYCAKHQVYWALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 451 aa, synthetic construct
EVQLVESGGGLVQPGGSLRLSCAASGFTFSDYAMSWVRQAPGKGLEWVSAISSSGGEKYYADSVKGRFTISRDNSKNTLYLQMNSLRAEDTAVYYCAKHGYGQEYYYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 451 aa, synthetic construct
EVQLVESGGGLVQPGGSLRLSCAASGFTFSSYAMSWVRQAPGKGLEWVSGISGSGSRTDYADSVKGRFTISRDNSKNTLYLQMNSLRAEDTAVYYCAKVYTYTRGFAFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 5 aa, synthetic construct
SYAMS

SEQ ID NO: 17 - protein, 5 aa, synthetic construct
DYAMS

SEQ ID NO: 18 - protein, 5 aa, synthetic construct
SYYMH

SEQ ID NO: 19 - protein, 5 aa, synthetic construct
DYAMS

SEQ ID NO: 20 - protein, 5 aa, synthetic construct
DYAMS

SEQ ID NO: 21 - protein, 5 aa, synthetic construct
DYAMS

SEQ ID NO: 22 - protein, 5 aa, synthetic construct
SYAMS

SEQ ID NO: 23 - protein, 17 aa, synthetic construct
AISSDGGSTYYADSVKG

SEQ ID NO: 24 - protein, 17 aa, synthetic construct
AISSSGGSTYYADSVKG

SEQ ID NO: 25 - protein, 17 aa, synthetic construct
RISPGGGGTIYAQKFQG

SEQ ID NO: 26 - protein, 17 aa, synthetic construct
RISSSGSSKYYADSVKG

SEQ ID NO: 27 - protein, 17 aa, synthetic construct
RISGSGGTKYYADSVKG

SEQ ID NO: 28 - protein, 17 aa, synthetic construct
AISSSGGEKYYADSVKG

SEQ ID NO: 29 - protein, 17 aa, synthetic construct
GISGSGSRTDYADSVKG

SEQ ID NO: 30 - protein, 10 aa, synthetic construct
RRLWSYSFDY

SEQ ID NO: 31 - protein, 10 aa, synthetic construct
LYYTYEVLDI

SEQ ID NO: 32 - protein, 12 aa, synthetic construct
AQSEGLSYYFDV

SEQ ID NO: 33 - protein, 14 aa, synthetic construct
WGSYGYGLVYYFDV

SEQ ID NO: 34 - protein, 9 aa, synthetic construct
HQVYWALDV

SEQ ID NO: 35 - protein, 12 aa, synthetic construct
HGYGQEYYYFDV

SEQ ID NO: 36 - protein, 12 aa, synthetic construct
VYTYTRGFAFDL

SEQ ID NO: 37 - protein, 214 aa, synthetic construct
DIQMTQSPSSLSASVGDRVTITCRASQSIGSYLNWYQQKPGKAPKLLIYAASTLQSGVPSRFSGSGSGTDFTLTISSLQPEDFATYYCQQSYSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 214 aa, synthetic construct
DIQMTQSPSSLSASVGDRVTITCRASQSISNYLNWYQQKPGKAPKLLIYAASRLQSGVPSRFSGSGSGTDFTLTISSLQPEDFATYYCQQSYSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 214 aa, synthetic construct
DIQMTQSPSSLSASVGDRVTITCRASQSIRNYLNWYQQKPGKAPKLLIYAASNLQSGVPSRFSGSGSGTDFTLTISSLQPEDFATYYCQQSSSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSESFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 214 aa, synthetic construct
DIQMTQSPSSLSASVGDRVTITCRASQSISNWLNWYQQKPGKAPKLLIYATSSLQSGVPSRFSGSGSGTDFTLTISSLQPEDFATYYCQQSYSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 214 aa, synthetic construct
DIQMTQSPSSLSASVGDRVTITCRASQSISNYLNWYQQKPGKAPKLLIYAASTLQSGVPSRFSGSGSGTDFTLTISSLQPEDFATYYCQQSYSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 214 aa, synthetic construct
DIQMTQSPSSLSASVGDRVTITCRASQSISNYLNWYQQKPGKAPKLLIYAASTLQSGVPSRFSGSGSGTDFTLTISSLQPEDFATYYCQQSYSFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 11 aa, synthetic construct
RASQSIGSYLN

SEQ ID NO: 45 - protein, 11 aa, synthetic construct
RASQSISNYLN

SEQ ID NO: 46 - protein, 11 aa, synthetic construct
RASQSIRNYLN

SEQ ID NO: 47 - protein, 11 aa, synthetic construct
RASQSISSYLN

SEQ ID NO: 48 - protein, 11 aa, synthetic construct
RASQSISNWLN

SEQ ID NO: 49 - protein, 11 aa, synthetic construct
RASQSISNYLN

SEQ ID NO: 50 - protein, 11 aa, synthetic construct
RASQSISNYLN

SEQ ID NO: 51 - protein, 7 aa, synthetic construct
AASTLQS

SEQ ID NO: 52 - protein, 7 aa, synthetic construct
AASRLQS

SEQ ID NO: 53 - protein, 7 aa, synthetic construct
AASNLQS

SEQ ID NO: 54 - protein, 7 aa, synthetic construct
AASSLQS

SEQ ID NO: 55 - protein, 7 aa, synthetic construct
ATSSLQS

SEQ ID NO: 56 - protein, 7 aa, synthetic construct
AASTLQS

SEQ ID NO: 57 - protein, 7 aa, synthetic construct
AASTLQS

SEQ ID NO: 58 - protein, 9 aa, synthetic construct
QQSYSFPWT

SEQ ID NO: 59 - protein, 9 aa, synthetic construct
QQSYSFPWT

SEQ ID NO: 60 - protein, 9 aa, synthetic construct
QQSSSFPLT

SEQ ID NO: 61 - protein, 9 aa, synthetic construct
QQSESFPLT

SEQ ID NO: 62 - protein, 9 aa, synthetic construct
QQSYSYPWT

SEQ ID NO: 63 - protein, 9 aa, synthetic construct
QQSYSTPWT

SEQ ID NO: 64 - protein, 9 aa, synthetic construct
QQSYSFPYT

SEQ ID NO: 65 - protein, 6 aa, synthetic construct
KGDEVD

SEQ ID NO: 66 - protein, 6 aa, synthetic construct
  SITE 1: note = 3H9-maleimide modified residue
  SITE 6: note = p-aminobenzylcarbamate-monomethyl auristatin E modified residue
KGDEVD

SEQ ID NO: 67 - protein, 6 aa, synthetic construct
KGDEVD

SEQ ID NO: 68 - protein, 6 aa, synthetic construct
  SITE 1: note = 3D1-maleimide modified residue
  SITE 6: note = p-aminobenzylcarbamate-monomethyl auristatin E modified residue
KGDEVD

SEQ ID NO: 69 - protein, 6 aa, synthetic construct
KGDEVD

SEQ ID NO: 70 - protein, 6 aa, synthetic construct
  SITE 1: note = 3D5-maleimide modified residue
  SITE 6: note = p-aminobenzylcarbamate-monomethyl auristatin E modified residue
KGDEVD

SEQ ID NO: 71 - protein, 6 aa, synthetic construct
KGDEVD

SEQ ID NO: 72 - protein, 6 aa, synthetic construct
  SITE 1: note = 4D1-maleimide modified residue
  SITE 6: note = p-aminobenzylcarbamate-monomethyl auristatin E modified residue
KGDEVD

SEQ ID NO: 73 - protein, 4 aa, synthetic construct
DEVD

SEQ ID NO: 74 - protein, 4 aa, synthetic construct
  SITE 1: note = 3H9-maleimide modified residue
  SITE 4: note = p-aminobenzylcarbamate-monomethyl auristatin E modified residue
DEVD

SEQ ID NO: 75 - protein, 6 aa, synthetic construct
KGDEVD

SEQ ID NO: 76 - protein, 6 aa, synthetic construct
  SITE 1: note = 3H9-maleimide modified residue
  SITE 6: note = p-aminobenzylcarbamate-Exatecan modified residue
KGDEVD

SEQ ID NO: 77 - protein, 6 aa, synthetic construct
  SITE 1: note = Maleimide modified residue
  SITE 6: note = p-aminobenzylcarbamate-monomethyl auristatin E modified residue
KGDEVD

SEQ ID NO: 78 - protein, 6 aa, synthetic construct
  SITE 1: note = Maleimide modified residue
KGDEVD

SEQ ID NO: 79 - protein, 4 aa, synthetic construct
  SITE 1: note = Maleimide modified residue
DEVD

(19) United States
(12) Patent Application Publication
(10) Pub. No.: US 2024/0091372 A1
(43) Pub. Date: Mar. 21, 2024

(54) ANTI-DOPPEL ANTIBODY DRUG CONJUGATES

(71) Applicants: SEOUL NATIONAL UNIVERSITY R&DB FOUNDATION, Seoul (KR); OSONG MEDICAL INNOVATION FOUNDATION, Chungcheongbuk-do (KR)

(72) Inventors: Youngro BYUN, Seoul (KR); Ha Kyeong LEE, Seoul (KR); Seungwoo CHUNG, Gyeonggi-do (KR); Byoung Mo KIM, Seoul (KR); So-Young CHOI, Sejong-si (KR); Se-Ra LEE, Sejong-si (KR)

(73) Assignees: SEOUL NATIONAL UNIVERSITY R&DB FOUNDATION, Seoul (KR); OSONG MEDICAL INNOVATION FOUNDATION, Chungcheongbuk-do (KR)

(21) Appl. No.: 18/354,476

(22) Filed: Jul. 18, 2023

Related U.S. Application Data
(60) Provisional application No. 63/368,860, filed on Jul. 19, 2022.

(51) Int. Cl. A61K 47/68; A61K 45/06; A61P 35/00; C07K 16/28

(52) U.S. Cl. CPC A61K 47/68031; A61K 45/06; A61K 47/68037; A61P 35/00; C07K 16/2872

(57) ABSTRACT

Described are anti-doppel antibody-drug conjugates, compositions comprising them, and related methods of treating doppel-associated diseases and conditions, including cancer.

### SEQUENCE LISTING

The instant application contains a Sequence Listing which has been submitted electronically in XML, file format and is hereby incorporated by reference in its entirety. Said XML copy, created on Jul. 13, 2023, is named 133143-0103_SL.xml and is 81,877 bytes in size.

### FIELD OF INVENTION

Described are anti-doppel antibody-drug conjugates, compositions comprising them, and related methods of treating doppel-associated diseases and conditions, including cancer.

### BACKGROUND

Chemotherapy is the most frequently applied anticancer therapy because of its powerful anticancer effect. Nevertheless the use of chemotherapy often is restricted because of serious side effects and toxicity. Efforts to develop selective chemotherapy that targets tumors as opposed to healthy tissue have focused on targeted delivery of chemotherapeutic agents to tumor cells, such as by using antibodies or peptides that recognize and bind to molecules expressed preferentially in tumor cells but not normal cells. Antibody-drug conjugates (ADCs) may comprise a potent cytotoxic drug and a target-specific antibody (e.g., an antibody that selectively binds a target-specific biomarker, such as a cancer-specific biomarker). ADCs may be described as a multistage rocket that consist of parts within parts that bring the payload (the cytotoxic drug) closer to its target (cancer cells). The primary targeting moiety of an ADC is the antibody. Once the antibody reaches and binds its target binding partner, effectively binding the ADC to the target binding partner, the ADC must be able to be internalized into the cell to exert its intended effect. For maximum efficacy, the cytotoxic drug must have a sufficient cytotoxicity to neutralize target cells in doses capable of delivery by the ADC.

The concept behind ADCs is deceptively simple, but it has proven extremely difficult to achieve sufficient efficacy of the approach in vivo. Despite progress made with ADC technology, ADC-based therapeutics still face challenges such as in vivo toxicity, suboptimal target biomarkers, unpredictable clinical value in combination therapies, and poorly understood pathways of drug resistance.

With regard to treatment of cancer, the identification of tumor-specific biomarkers has changed the way we view cancer. Cancer is no longer considered a single disease. Because a cancer may be the result of accumulated mutations, a single cancer may be divided into multiple subtypes, each with its own set of identifying mutations. Conversely, tumors from completely different parts of the body can have similar mutations and respond to the same medications. See, e.g., Sun et al., Bioconjugate Chem. 2020, 31 (4): 1012-1024. Therefore, it has been increasingly important in ADC-based chemotherapies to identify and quantify tumor-specific antigens for use as target-specific biomarkers. Although there has been a surge in the discovery of tumor-specific and tumor-associated antigens, to be safely and effectively used in ADC biomarkers should meet several requirements, such as being highly expressed on the tumor surface, conducive to internalization of the ADC, and having limited expression on normal tissues (to prevent off-target toxicities). In this regard, although tumor-associated antigens for solid tumors such as HER2, TROP2 and nectin 4 have been used to develop monoclonal antibodies for therapeutic and chemotherapeutic use, these tumor-associated antigens also are expressed in large amounts in normal tissues, which can lead to undesired targeting of healthy tissue (off-target toxicity).

Thus, there remains a need for safe and effective ADCs that selectively bind to their target (e.g., tumor cells), and do not harm non-target cells (e.g., normal tissue) but are effective against their target cells (e.g., tumor cells).

### SUMMARY

Provided herein are anti-doppel antibody drug conjugates (ADCs) comprising: (i) a doppel-targeting moiety, (ii) a cleavable linker, and (iii) a therapeutic agent. Also provided herein are methods of treating doppel-associated diseases and conditions, including doppel-associated cancers, using the ADCs described herein and kits comprising the ADCs describe herein.

In some aspects, there are provided anti-doppel ADCs comprising: (i) a doppel-targeting moiety, joined directly or through a linker to (ii) a cleavable linker, joined directly or through a linker to (iii) a therapeutic agent.

In some aspects, the doppel-targeting moiety is selected from a doppel-binding monoclonal antibody, a doppel-binding polyclonal antibody, a doppel-binding single chain antibody, a doppel-binding chimeric antibody, a doppel-binding humanized antibody, a doppel-binding veneered antibody, and doppel-binding fragments of any thereof. In some aspects, the doppel-targeting moiety is a doppel-binding antibody selected from human monoclonal antibody A12 disclosed herein; human monoclonal antibody B2 disclosed herein; human monoclonal antibody E9 disclosed herein; human monoclonal antibody 3D5 disclosed herein; human monoclonal antibody 3D1 disclosed herein; human monoclonal antibody 4D1 disclosed herein; human monoclonal antibody 3H9 disclosed herein, and doppel-binding fragments of any thereof.

In some aspects, the cleavable linker is cleavable by an intracellular protease. In some aspects, the cleavable linker is selected from a dipeptide cleavable linker such as valine-citrulline, valine-alanine, and phenylalanine-lysine; a hydrazone linker hydrolyzed at a pH of less than 5.5, and a disulfide linker cleaved in a reduced environment.

In some aspects, the cleavable peptide linker is a caspase-cleavable peptide linker. In some aspects, the four C-terminal amino acid residues of the caspase-cleavable peptide linker are selected from Asp-Xaa-Xaa-Asp, Leu-Xaa-Xaa-Asp, and Val-Xaa-Xaa-Asp, where Xaa represents any amino acid residue. In some aspects, the four C-terminal amino acid residues of the caspase-cleavable peptide linker are selected from Asp-Glu-Val-Asp (SEQ ID NO:4), Asp-Leu-Val-Asp (SEQ ID NO:5) Asp-Glu-Ile-Asp (SEQ ID NO:6), and Leu-Glu-His-Asp (SEQ ID NO:7). In some aspects, the six C-terminal amino acid residues of the caspase-cleavable peptide linker consist of Lys-Gly-Asp-Glu-Val-Asp (SEQ ID NO:8).

In some aspects, the therapeutic agent comprises a chemotherapeutic agent. In some aspects, the therapeutic agent comprises a chemotherapeutic agent that induces apoptosis of tumor cells. In some aspects the therapeutic agent is selected from 5-FU, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, exatecan, camptothecin, carboplatin, 10-hydroxycamptothecin, carmustine, celebrex, chlorambucil, cisplatin, COX-2 inhibitors, irinotecan, SN-38, cladribine, crizotinib, cyclophosphamide, cytarabine, dacarbazine, dasatinib, dinaciclib, docetaxel, dactinomycin, daunorubicin, doxorubicin, epidophyllotoxin, erlotinib, entinostat, estrogen receptor binding agents, etoposide, exemestane, fingolimod, floxuridine, fludarabine, flutamide, flavopiridol, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechloresthamine, melphalan, mercaptopurine, methotrexate, mitoxantrone, mithramycin, mitomycin, mitotane, navelbine, neratinib, nilotinib, nitrosurea, olaparib, plicomycin, procarbazine, paclitaxel, PCI-32765, pentostatin, PSI-341, raloxifene, semustine, sorafenib, streptozocin, SU11248, sunitinib, tamoxifen, temazolomide, transplatin, thalidomide, thioguanine, thiotepa, teniposide, topotecan, uracil mustard, vatalanib, vinorelbine, vinblastine, vincristine, vinca alkaloids and ZD183. In some aspects, the chemotherapeutic agent is selected from anthracyclines, antibiotics, alkylating agents, platinum-based agents, antimetabolites, topoisomerase inhibitors, and mitotic inhibitors. In some aspects, the chemotherapeutic agent is selected from the group consisting of doxorubicin, daunorubicin, epirubicin, idarubicin, valrubicin, and derivatives thereof. In other aspects, the chemotherapeutic agent is selected from actinomycin-D, bleomycin, mitomycin-C, calicheamicin, and derivatives thereof. In still other aspects, the chemotherapeutic agent is selected from cyclophosphamide, mechlorethamine, uramustine, melphalan, chlorambucil, ifosfamide, bendamustine, carmustine, lomustine, streptozocin, busulfan, dacarbazine, temozolomide, thiotepa, altretamine, duocarmycin, cisplatin, carboplatin, nedaplatin, oxaliplatin, satraplatin, triplatin tetranitrate, 5-fluorouracil, 6-mercaptopurine, capecitabine, cladribine, clofarabine, cystarbine, floxuridine, fludarabine, gemcitabine, hydroxyurea, methotrexate, pemetrexed, pentostatin, thioguanine, exatecan, camptothecin, topotecan, irinotecan, etoposide, teniposide, mitoxantrone, paclitaxel, docetaxel, izabepilone, vinblastine, vincristine, vindesine, vinorelbine, estramustine, maytansine, DM1 (mertansine), DM4, dolastatin, auristatin E, auristatin F, monomethyl auristatin E (MMAE) and derivatives thereof. In some aspects the chemotherapeutic agent is selected from monomethyl auristatin E (MMAE) and derivatives thereof.

In some aspects, the therapeutic agent comprises an immunomodulatory agent. In some aspects, the immunomodulatory agent is selected from cytokines, lymphokines, monokines, stem cell growth factors, lymphotoxins, hematopoietic factors, colony stimulating factors (CSF), interrerons (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, transforming growth factor (TGF), TGF-alpha, TGF-beta, insulin-like growth factor (IGF), erythropoietin, thrombopoietin, tumor necrosis factor (TNF), TNF-alpha, TNF-beta, mullerian-inhibiting substance, mouse gonadotropin-associated peptide, inhibin, activin, vascular endothelial growth factor, integrin, interleukin (IL), granulocyte-colony stimulating factor (G-CSF), granulocyte macrophage-colony stimulating factor (GM-CSF), interferon-alpha, interferon-beta, interferon-gamma, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-23, IL-25, LIF, kit-ligand, FLT-3, angiostatin, thrombospondin, endostatin, toll-like receptor (TLR) agonists (such as CU-T12-9, Pam3CSK4, FSL-1, Pam2CSK4, and CL429), Poly(A:U), Poly(I:C), lipopolysaccharides (LPS), MPLA-SM, CRX-527, flagellin, thiazoquinoline derivatives, imidazoquinoline derivatives (such as CL097, gardiquimod, imiquimod, and resiquimod), adenine analogs, guanosine analogs, thymidine analogs, benzoazepine analogs, and CpG oligodeoxynucleotides (ODN) (such as ODN 1585, ODN 2216, ODN 2336, ODN 1668, ODN 1826, ODN 2006, ODN 2007, ODN BW006, ODN D-SL01, ODN 2395, ODN M362, and ODN D-SL03).

In some aspects, the therapeutic agent comprises a toxin. In some aspects, the toxin is selected from ricin, abrin, ribonuclease (RNase), DNase 1, Staphylococcal enterotoxin-A, pokeweed antiviral protein, gelonin, diphtheria toxin, Pseudomonas exotoxin, and Pseudomonas endotoxin.

In some aspects, the therapeutic agent comprises a radionuclide. In some aspects, radionuclide is selected from the 11C, 13N, 15O, 32P, 33P, 47Sc, 51Cr, 57Co, 58Co, 59Fe, 62Cu, 67Cu, 67Ga, 75Br, 75Se, 76Br, 77As, 77Br, 80mBr, 89Sr, 90Y, 95Ru, 97Ru, 99Mo, 99mTc, 103mRh, 103Ru, 105Rh, 105Ru, 107Hg, 109Pd, 109Pt, 111Ag, 111In, 113mIn, 119Sb, 121mTe, 122mTe, 125I, 125mTe, 126I, 131I, 133I, 142Pr, 143Pr, 149Pm, 152Dy, 153Sm, 161Ho, 161Tb, 165Tm, 166Dy, 166Ho, 167Tm, 168TM, 169Er, 169Yb, 177Lu, 186Re, 188Re, 189mOs, 189Re, 198Ir, 194Ir, 197Pt, 198Au, 199Au, 203Hg, 211At, 211Bi, 211Pb, 212Bi, 212Pb, 213Bi, 215Po, 217At, 219Rn, 221Fr, 223Ra, 225Ac, 227Th and 255Fm.

In some aspects, the therapeutic agent is an DNA cross-linking agent, such as one or more selected from indolionobenzodiazepine dimer (IGN), pyrrolobenzodiazepine (PBD), and derivatives thereof.

In a specific embodiment, the doppel-targeting moiety is a doppel-targeting antibody, the linker is a caspase-cleavable peptide linker having an amino acid sequence consisting of Lys-Gly-Asp-Glu-Val-Asp (SEQ ID NO:8), and the chemotherapeutic agent is MMAE.

In a further specific embodiment, the doppel-targeting moiety is human monoclonal antibody 3H9, the linker is a caspase-cleavable peptide linker having an amino acid sequence consisting of Lys-Gly-Asp-Glu-Val-Asp (SEQ ID NO:8), and the chemotherapeutic agent is MMAE.

Also provided are compositions comprising an ADC as described herein, and a pharmaceutically acceptable carrier. In some aspects, the composition is formulated for intravenous administration.

Also provided are methods of treating doppel-associated diseases or conditions in a subject, comprising administering to a subject in need thereof an ADC as described herein.

In some aspects, the doppel-associated disease or condition is selected from asthma, tuberculosis, atherosclerosis, and pulmonary arterial hypertension (PAH).

In some aspects, the doppel-associated disease or condition is cancer. In some aspects, the cells of the cancer express doppel.

Also provided are methods of treating a doppel-associated cancer in a subject, comprising administering to a subject in need thereof an ADC as described herein.

Also provided are kits comprising an ADC as described herein contained in a suitable container, and optionally further comprising instructions for use.

The foregoing summary is illustrative only and is not intended to be in any way limiting. In addition to the illustrative aspects, embodiments, and features described above, further aspects, embodiments, and features will become apparent by reference to the detailed description and examples herein below.

### BRIEF DESCRIPTION OF THE DRAWINGS

FIG. 1 illustrates the cytotoxicity of an anti-doppel ADC as described herein named 3H9-KGDEVD-MMAE (name=SEQ ID NO:65; structure=SEQ ID NO:66) on HCT116 cells.

FIG. 2 illustrates the internalization of 3H9 monoclonal antibody (mAb) into doppel-expressing cells (HCTEC) observed by confocal microscopy.

FIG. 3 illustrates the lysosomal colocalization of 3H9 mAb in doppel-expressing cells (HCTEC) observed by confocal microscopy.

FIG. 4 illustrates the in vivo efficacy of anti-doppel ADCs as described herein (3D1-vc-MMAE; 3D5-vc-MMAE; 3H9-vc-MMAE; and 4D1-vc-MMAE).

FIG. 5 illustrates the dose-dependency of anti-doppel ADCs as described herein (3D1-vc-MMAE; 3D5-vc-MMAE; and 3H9-vc-MMAE).

FIG. 6 illustrates the in vivo efficacy of anti-doppel ADCs as described herein having different linkers (3H9-KGDEVD-MMAE (name=SEQ ID NO:65; structure=SEQ ID NO:66); 3H9-DEVD-MMAE (name=SEQ ID NO:73; structure=SEQ ID NO:74), and 3H9-vc-MMAE).

FIG. 7 illustrates the in vivo efficacy of anti-doppel ADCs as described herein (3D1-KGDEVD-MMAE (name=SEQ ID NO:67; structure=SEQ ID NO:68); 3D5-KGDEVD-MMAE (name=SEQ ID NO:69; structure=SEQ ID NO:70); 3H9-KGDEVD-MMAE (name=SEQ ID NO: 65; structure=SEQ ID NO:66); and 4D1-KGDEVD-MMAE (name=SEQ ID NO:71; structure=SEQ ID NO:72)).

### DETAILED DESCRIPTION

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention based on the guidance provided herein. However, specific materials and methods are described for illustrative purposes. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth herein, and is intended to refer to ranges substantially around the number without departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular number.

As used herein, the term “tumor cell(s)” refers to cells of any type of tumor tissue, benign or malignant.

As used herein, the term “cancer” refers to cancer originating from any part of the body or any cell type. This includes, but is not limited to, carcinoma, sarcoma, lymphoma, germ cell tumors, and blastoma. The cancer may be associated with a specific location in the body or a specific disease. The terms “cancer” and “tumor” generally are used interchangeably.

As used herein, unless the context dictates otherwise, the term “cell” includes cells in animals (e.g., in vivo cells) and cultured cells.

Unless otherwise stated, as used herein “doppel” refers to any doppel protein, including doppel protein of mouse or human origin, including doppel protein in glycosylated or aglycosylated (not glycosylated) form, including doppel in monomeric or dimeric form. The terms “doppel,” “PRND,” and “doppel protein” generally are used interchangeably herein.

As used herein, the term “subject” refers to any animal in need of treatment by any one or more of the methods described herein, including humans and other mammals, such as dogs, cats, rabbit, horses, and cows. For example, a subject may be suffering from or at risk of developing a disease or condition associated with doppel, including a doppel-associated cancer. In specific aspects, the subject is a human diagnosed with a doppel-associated tumor or a doppel-associated cancer.

As used herein, the phrase “disease or condition associated with doppel” (or “doppel-associated disease or condition”) refers to a disease or condition in which the subject's endothelial cells express doppel, including asthma, tuberculosis, atherosclerosis, pulmonary arterial hypertension (PAH), and neoplasms and neoplasm-related conditions, including doppel-associated cancers and doppel-associated tumors. As used herein, the terms “doppel-associated” tumor and “doppel-associated” cancer refer to a tumor or cancer, the cells of which express doppel.

### Anti-Doppel Antibody-Drug Conjugates

Described herein are anti-doppel antibody-drug conjugates (ADCs) comprising: (i) a doppel-targeting moiety, (ii) a cleavable linker, and (iii) a therapeutic agent. Also described herein are methods of treating doppel-associated diseases and conditions, including doppel-associated cancers, using the ADCs described herein. As used herein, nomenclature such as [antibody name]-cleavable linker-[therapeutic agent name] is used to designate ADCs comprising the named antibody, cleavable linker, and therapeutic agent. It should be understood the other moieties may be present, such as one or more other chemical linking agents, as discussed in more detail below and illustrated in the examples.

As discussed in more detail below, the anti-doppel ADCs and methods described herein build on the inventors' discovery that doppel is a tumor endothelial cell (TEC) surface marker that plays a role in pathological angiogenesis, and that inhibiting doppel angiogenetic activity, such as by inhibiting the interaction of doppel with a tyrosine kinase receptor (e.g. VEGFR2), such as by binding doppel or otherwise, can selectively inhibit pathological angiogenesis, including tumor angiogenesis and pathological angiogenesis associated with other conditions associated with doppel-expressing endothelial cells, such as asthma, tuberculosis, atherosclerosis, pulmonary arterial hypertension (PAH), neoplasms, and neoplasm-related conditions. For example, doppel expression on endothelial cells may be increased during pathological angiogenesis relative to its expression during normal or physiological angiogenesis conditions. Additionally, doppel expression on TECs may be associated with pathological tumor-associated angiogenesis or tumorigenesis.

### Doppel

Doppel is a prion-like protein encoded by a gene, PRND, which is located near the PRNP (Prion protein coding gene) locus. See, e.g., Golaniska et al., Folia Neuropathol, 42 (Supp. A) 47-54 (2004). Doppel expression is conserved through evolution from humans to mice, which indicates that doppel expression may play an essential function under certain physiological conditions. See, e.g., Behrens et al., EMBO J. 21:3652 (2002). Full-length human doppel is a 179 amino acid residue protein (UniProtKB Q9UKYO; NCBI Ref NP 036541.2) with a molecular weight of 14 kDa for the non-glycosylated form. Doppel undergoes a C-terminal glycosylphosphatidylinositol (GPI) modification and is expressed on the cell surface anchored to a lipid raft by GPI.

Doppel is transiently expressed in the brain endothelium of neonates, but in adults, it is expressed only in testicular cells. (Li et al., Am. J. Pathol. 2000; 157(5):1447-1452.) According to the human protein atlas, PRND is nearly nonexistent outside the gonads in normal human adults. Due to the lack of doppel on most normal cells, anti-doppel ADCs as described herein offer a highly target-specific approach to ADC targeting that is not possible with other tumor biomarkers. Therefore, the methods described herein will have limited off-target toxicities, offering a significant improvement over current approaches.

On the other hand, doppel is associated with various diseases and conditions for which safe and effective therapies are still needed. For example, doppel has been implicated in neurodegeneration and angiogenesis. Importantly for the context of the present disclosure, doppel is highly expressed in tumors. The most extensive study on the relationship between malignant tissue and doppel expression was done by Comincini et al. (Anticancer Research, 2004, 24:1507-1518) in astrocytoma, who studied PRND expression in glioblastoma-derived cell lines and non-glial tumor specimens, and found PRND expression to be directly related to tumor malignancy. The authors reported immunohistochemical analysis revealing a diffuse cytoplasmatic doppel distribution in different astrocytic neoplastic cells, in infiltrating lymphocytes, and in blood vessel endothelial cells. The authors also reported high levels of PRND in non-glial malignant tumor samples, such as gastric adenocarcinoma and anaplastic meningioma, suggesting that doppel could act as a biomarker for other forms of cancer in addition to glioblastoma. Al-Hilal et al. (J. Clin. Invest. 2016, 126 (4): 1251-66) reported a study on doppel as a potential therapeutic target for tumor angiogenesis, and found doppel expression in tumor endothelial cells (TECs), but not normal endothelial cells (ECs), and reported that blocking doppel could potentially selectively inhibit tumor angiogenesis.

For the context of the present disclosure, the localization of doppel on the surface of TECs make it a superior target for ADC compared to targets on the tumor itself for several reasons.

First, TECs are localized in the blood vessel and constitute the first cell layer encountered by therapeutics administered intravenously. Thus, in some aspects, anti-doppel ADCs are, in effect, vascular-targeting ADCs (VT-ADCs). One advantage of VT-ADCs over ADCs that target other biomarkers is the proximity of the target to the blood vessel. In conventional chemotherapy targeting other biomarkers, the chemotherapeutic agent has to overcome multiple barriers in order to reach its target. For example, even before it reaches the cell, it has to pass through the interstitial exocellular matrix (ECM) of the tumor, which presents diverse barriers to the chemotherapeutic agent. For example, tumor ECMs are hypertensive by nature due to leaky vessels and blocked lymph nodes. This interstitial hypertension suppresses extravasation and intestinal transport of macromolecules. Further complicating target localization is the dense network of collagen fiber saturating the ECM, as well as the increased distance between the tumor cells. In this regard, the relative volume of interstitial space is about 3-5 time larger than normal cells, extending the length the chemotherapeutic agent has to travel. (Kratz, F. et al., Drug delivery in oncology, (2011) John Wiley & Sons, Ltd. pp. 40-44) Furthermore, even if the chemotherapeutic agent reaches the center of the tumor, its efficacy may be limited by the nature of its design. Most chemotherapeutic agents target the rapid proliferation of tumor cells. However, malignant cells distant from blood vessels are in a state of quiescence because of unfavorable conditions and so may not respond to the chemotherapeutic agent. (Kratz, F. et al., Drug delivery in oncology, (2011) John Wiley & Sons, Ltd. p. 15) In contrast, anti-doppel ADCs as described herein targeting doppel on the surface of TECs do not have to travel through the intestinal space, and therefore avoid the problems outlined above. This not only makes the ADCs more effective against their target, but also limits off-target toxicities and provides a more predictable PK/PD relationship. Furthermore, tumors of all types rely on angiogenesis if they are to reach a size larger than 1 cm³. Therefore, anti-doppel ADCs as described herein can be effective against the majority of the tumor mass (e.g., including tumor cells distant from blood vessels) due to loss of vasculature induced by the chemotherapeutic agent carried by the anti-doppel ADCs described herein.

Another advantage of targeting doppel in view of the localization of doppel on the surface of TECs relates to tumor heterogeneity. Even within the same tumor, cells may express different biomarkers and mutations are frequent. This may be because the hypoxic environment within tumors can cause further changes in the proteome and genome, driving tumor evolution toward tumor progression, including chemotherapeutic drug resistance. (Kratz, F. et al., Drug delivery in oncology, (2011) John Wiley & Sons, Ltd., p. 49) In contrast, TECs are of normal hematopoietic origins and are less likely to undergo somatic mutation.

As noted above, doppel is not only selectively expressed on tumor cells but also on endothelial cells under pathological conditions, such as atherosclerosis, tuberculosis, asthma, and pulmonary arterial hypertension (PAH). Although the role of doppel in the development of these diseases is not fully understood, the doppel protein has been identified as a potential target for treating these diseases. As doppel is actively involved in angiogenic signals, targeting doppel can be a promising way to selectively inhibit pathological angiogenesis.

### Doppel-Targeting Moiety

As noted above, the anti-doppel ADCs described herein comprise a doppel-targeting moiety. In some aspects, the doppel-targeting moiety is an anti-doppel antibody that binds doppel, or a related species, such as a doppel-binding antibody fragment, including, but not limited to, an antibody fragment or peptide that binds to doppel. In some aspects, the binding inhibits interaction of doppel with a tyrosine kinase receptor, such as one or more of VEGFR2, VEGFR1, VEGFR3, bFGFR, and PDGFR. In some aspects, the doppel-targeting moiety binds to doppel and thereby inhibits the interaction of doppel with VEGFR2. In some embodiments, the anti-doppel antibody is directed against the extracellular domain (ECD) of doppel.

As used herein the term “antibody” or “anti-doppel antibody” as used herein includes a monoclonal antibody, a polyclonal antibody, a recombinant antibody, a humanized antibody (see, e.g., Jones et al., Nature 321 (1986), 522-525; Riechmann et al., Nature 332 (1988), 323-329; Presta, Curr. Op. Struct. Biol. 2 (1992), 593-596), a chimeric antibody (see, e.g., Morrison et al., Proc. Natl. Acad. Sci. U.S.A. 81 (1984), 6851-6855), a human antibody, a fully humanized antibody (see, e.g., Tomizuka et al., Nature Genetics (1997) 16, 133-143; Kuroiwa, et al., Nucl. Acids Res. (1998) 26, 3447-3448; Yoshida, H. et al., ANIMAL CELL TECHNOLOGY: BASIC AND APPLIED ASPECTS Vol. 10, 69-73 (Kitagawa, Y., Matsuda, T. and Iijima, S. eds.) (Kluwer Academic Publishers, 1999); Tomizuka, et al., Proc. Natl. Acad. Sci. USA (2000) 97, 722-727, WO 2007/077028), a multispecific antibody (e.g., a bispecific antibody) formed from at least two antibodies, or an antibody fragment of any thereof.

As used herein the term “doppel-binding antibody fragment” refers to any portion of the afore-mentioned types antibodies capable of binding to doppel, typically comprising an antigen-binding region or variable regions. Examples of antibody fragments include Fab fragments, Fab ‘ fragments, F(ab’)2 fragments, Fv fragments, diabodies (see, e.g., Hollinger et al., Proc. Natl. Acad. Sci. U.S.A. 90 (1993), 6444-6448), single-chain antibody molecules (see, e.g., Pluckthun in: THE PHARMACOLOGY OF MONOCLONAL ANTIBODIES 113 (Rosenburg and Moore, eds.) (Springer Verlag, N.Y. (1994), 269-315), and other fragments that exhibit binding to doppel. Thus, an antibody fragment useful as a doppel-targeting moiety of the ADCs described herein may comprise a portion of a full-length antibody, such as its antigen-binding domain or variable region domain. Examples of suitable antibody fragments include Fab, F(ab′)2, Fv, and single-chain Fv (scFv) constructs (in which Fv fragments from the heavy and light chains are ligated by an appropriate linker) (see, e.g., Huston et al., Proc. Natl. Acad. Sci. U.S.A. 85:5879-5883 (1988); diabodies; linear antibodies; single-chain antibody molecules; and multispecific antibodies formed from antibody fragments.

As used herein, the term “diabodies” refers to small antibody fragments with two antigen-binding sites, which fragments comprise a heavy chain variable domain (VH) connected to a light chain variable domain (VL) in the same polypeptide chain (VH-VL) by a linker. The linker is too short to allow pairing between the two domains on the same chain so that the domains are forced to pair with complementary domains of another chain and create two antigen-binding sites. Diabodies are described more fully in, for example, EP 404 097; WO 93/11161; and Hollinger et al., Proc. Natl. Acad. Sci, USA 90:6444-6448 (1993).

In addition, the term “antibody” or “anti-doppel antibody” as used herein may include antibody-like molecules that contain engineered sub-domains of antibodies or naturally occurring antibody variants. These antibody-like molecules may be single-domain antibodies such as VH-only or VL-only domains derived either from natural sources such as camelids (see, e.g., Muyldermans et al., J. Biotech. 2001, 74, 277-302) or through in vitro display of libraries from humans, camelids or other species (see, e.g., Holt et al., Trends Biotechnol., 2003, 21, 484-90).

The term “monoclonal antibody” as used herein refers to an antibody obtained by a single clone of B-lymphocytes or by a cell into which the light and heavy chain genes of a single antibody have been transfected. Monoclonal antibodies are highly specific, being directed against a single antigenic site. Furthermore, in contrast to polyclonal antibody preparations, which typically include multiple antibodies directed against different determinants (epitopes), each monoclonal antibody is directed against a single determinant (epitope) on the antigen.

Thus, the doppel-targeting moiety of an ADC as described herein may be any antibody or antibody-like molecule, including, but not limited to, a polyclonal antibody or a monoclonal antibody, or a derivative of an antibody, such as a single chain antibody, a chimeric antibody, a humanized antibody (or other species-ized antibody modified for use in another target species), a veneered antibody, etc. The antibody may be glycosylated or aglycosylated, or have a modified glycosylation pattern.

Doppel-targeting moieties suitable for use in the ADCs and methods described herein include the doppel-targeting molecules described in U.S. patent application Ser. No. 17/350,763 filed Jun. 17, 2021, the entire contents of which are incorporated herein by reference.

The general structure of antibodies is known in the art and will only be briefly summarized here. An immunoglobulin monomer comprises two heavy chains and two light chains connected by disulfide bonds. Each heavy chain is paired with one of the light chains to which it is directly bound via a disulfide bond. Each heavy chain comprises a constant region (which varies depending on the isotype of the antibody) and a variable region. The variable region comprises three hypervariable regions (or complementarity determining regions) which are designated CDRH1, CDRH2 and CDRH3 and which are supported within framework regions. Each light chain comprises a constant region and a variable region, with the variable region comprising three hypervariable regions (designated CDRL1, CDRL2 and CDRL3) supported by framework regions in an analogous manner to the variable region of the heavy chain.

The hypervariable regions of each pair of heavy and light chains mutually cooperate to provide an antigen binding site that is capable of binding a target antigen. The binding specificity of a pair of heavy and light chains is defined by the sequence of their respective CDRs. Thus once a set of CDR sequences (i.e., the sequence of the three CDRs for the heavy and light chains) is determined which gives rise to a particular binding specificity, the set of CDR sequences can, in principle, be inserted into the appropriate positions within any other antibody framework regions linked with any antibody constant regions in order to provide a different antibody with the same antigen binding specificity.

In some embodiments, the doppel-targeting moiety is an anti-doppel antibody. In specific embodiments, the doppel-targeting moiety is a human monoclonal antibody selected from human monoclonal antibody A12, human monoclonal antibody B2, human monoclonal antibody E9, human monoclonal antibody 3D5, human monoclonal antibody 3D1, human monoclonal antibody 4D1, human monoclonal antibody 3H9, and doppel-binding fragments of any thereof. In specific embodiments, the doppel-targeting moiety has an amino acid sequence that is at least 85%, 90%, 95%, or 99% identical to monoclonal antibody A12, human monoclonal antibody B2, human monoclonal antibody E9, human monoclonal antibody 3D5, human monoclonal antibody 3D1, human monoclonal antibody 4D1, human monoclonal antibody 3H9.

In some embodiments, the doppel-targeting moiety is an anti-doppel antibody having CDR sequences that are at least 85%, 90%, 95%, 99%, or 100% identical to the CDRs of human monoclonal antibody A12, human monoclonal antibody B2, human monoclonal antibody E9, human monoclonal antibody 3D5, human monoclonal antibody 3D1, human monoclonal antibody 4D1, or human monoclonal antibody 3H9.

In some embodiments, the doppel-targeting moiety is an anti-doppel antibody having heavy chain sequences that are at least 85%, 90%, 95%, 99%, or 100% identical to the heavy chain sequence of human monoclonal antibody A12 (SEQ ID NO:9), heavy chain sequence of human monoclonal antibody B2 (SEQ ID NO:10), heavy chain sequence of human monoclonal antibody E9 (SEQ ID NO:11), heavy chain sequence of human monoclonal antibody 3D5 (SEQ ID NO:12), heavy chain sequence of human monoclonal antibody 3D1 (SEQ ID NO:13), heavy chain sequence of human monoclonal antibody 4D1 (SEQ ID NO:14), or human monoclonal antibody 3H9 (SEQ ID NO:15). In some embodiments, the doppel-targeting moiety is an anti-doppel antibody having heavy chain variable domain sequences that are at least 85%, 90%, 95%, 99%, or 100% identical to the heavy chain variable domain sequence of human monoclonal antibody A12, human monoclonal antibody B2, human monoclonal antibody E9, human monoclonal antibody 3D5, human monoclonal antibody 3D1, human monoclonal antibody 4D1, or human monoclonal antibody 3H9. In specific embodiments, the heavy chain variable region comprises a CDRH1 amino acid sequence at least 85%, 90%, 95%, 99%, or 100% identical to the CDRH1 sequence of any one of human monoclonal antibody A12 CDRH1 (SEQ ID NO:16), human monoclonal antibody B2 CDRH1 (SEQ ID NO:17), human monoclonal antibody E9 CDRH1 (SEQ ID NO:18), human monoclonal antibody 3D5 CDRH1 (SEQ ID NO:19), human monoclonal antibody 3D1 CDRH1 (SEQ ID NO:20), human monoclonal antibody 4D1 CDRH1 (SEQ ID NO:21), or human monoclonal antibody 3H9 CDRH1 (SEQ ID NO:22). In specific embodiments, the heavy chain variable region comprises a CDRH2 amino acid sequence at least 85%, 90%, 95%, 99%, or 100% identical to the CDRH2 sequence of any one of human monoclonal antibody A12 CDRH2 (SEQ ID NO:23), human monoclonal antibody B2 CDRH2 (SEQ ID NO:24), human monoclonal antibody E9 CDRH2 (SEQ ID NO:25), human monoclonal antibody 3D5 CDRH2 (SEQ ID NO:26), human monoclonal antibody 3D1 CDRH2 (SEQ ID NO:27), human monoclonal antibody 4D1 CDRH2 (SEQ ID NO:28), or human monoclonal antibody 3H9 CDRH2 (SEQ ID NO:29). In specific embodiments, the heavy chain variable region comprises a CDRH3 amino acid sequence at least 85%, 90%, 95%, 99%, or 100% identical to the CDRH3 sequence of any one of human monoclonal antibody A12 CDRH3 (SEQ ID NO:30), human monoclonal antibody B2 CDRH3 (SEQ ID NO:31), human monoclonal antibody E9 CDRH3 (SEQ ID NO:32), human monoclonal antibody 3D5 CDRH3 (SEQ ID NO:33), human monoclonal antibody 3D1 CDRH3 (SEQ ID NO:34), human monoclonal antibody 4D1 CDRH3 (SEQ ID NO:35), or human monoclonal antibody 3H9 CDRH3 (SEQ ID NO:36).

In some embodiments, the doppel-targeting moiety is an anti-doppel antibody having light chain sequences that are at least 85%, 90%, 95%, 99%, or 100% identical to the light chain sequence of human monoclonal antibody A12 (SEQ ID NO:37), the light chain sequence of human monoclonal antibody B2 (SEQ ID NO:38), the light chain sequence of human monoclonal antibody E9 (SEQ ID NO:39), the light chain sequence of human monoclonal antibody 3D5 (SEQ ID NO:40), the light chain sequence of human monoclonal antibody 3D1 (SEQ ID NO:41), the light chain sequence of human monoclonal antibody 4D1 (SEQ ID NO:42), or human monoclonal antibody 3H9 (SEQ ID NO:43). In some embodiments, the doppel-targeting moiety is an anti-doppel antibody having light chain variable domain sequences that are at least 85%, 90%, 95%, 99%, or 100% identical to the light chain variable domain sequence of human monoclonal antibody A12, human monoclonal antibody B2, human monoclonal antibody E9, human monoclonal antibody 3D5, human monoclonal antibody 3D1, human monoclonal antibody 4D1, or human monoclonal antibody 3H9. In specific embodiments, the light chain variable region comprises a CDRL1 amino acid sequence at least 85%, 90%, 95%, 99%, or 100% identical to the CDRL1 sequence of any one of human monoclonal antibody A12 CDRL1 (SEQ ID NO:44), human monoclonal antibody B2 CDRL1 (SEQ ID NO:45), human monoclonal antibody E9 CDRL1 (SEQ ID NO:46), human monoclonal antibody 3D5 CDRL1 (SEQ ID NO:47), human monoclonal antibody 3D1 CDRL1 (SEQ ID NO:48), human monoclonal antibody 4D1 CDRL1 (SEQ ID NO:49), or human monoclonal antibody 3H9 CDRL1 (SEQ ID NO:50). In specific embodiments, the light chain variable region comprises a CDRL2 amino acid sequence at least 85%, 90%, 95%, 99%, or 100% identical to the CDRL2 sequence of any one of human monoclonal antibody A12 CDRL2 (SEQ ID NO:51), human monoclonal antibody B2 CDRL2 (SEQ ID NO:52), human monoclonal antibody E9 CDRL2 (SEQ ID NO:53), human monoclonal antibody 3D5 CDRL2 (SEQ ID NO:54), human monoclonal antibody 3D1 CDRL2 (SEQ ID NO:55), human monoclonal antibody 4D1 CDRL2 (SEQ ID NO:56), or human monoclonal antibody 3H9 CDRL2 (SEQ ID NO:57). In specific embodiments, the light chain variable region comprises a CDRL3 amino acid sequence at least 85%, 90%, 95%, 99%, or 100% identical to the CDRL3 sequence of any one of human monoclonal antibody A12 CDRL3 (SEQ ID NO:58), human monoclonal antibody B2 CDRL3 (SEQ ID NO:59), human monoclonal antibody E9 CDRL3 (SEQ ID NO:60), human monoclonal antibody 3D5 CDRL3 (SEQ ID NO:61), human monoclonal antibody 3D1 CDRL3 (SEQ ID NO:62), human monoclonal antibody 4D1 CDRL3 (SEQ ID NO:63), or human monoclonal antibody 3H9 CDRL3 (SEQ ID NO:64).

In some embodiments, the doppel-targeting moiety is an anti-doppel antibody having framework region sequence that are at least 85%, 90%, 95%, 99%, or 100% identical to the framework sequence of any one of human monoclonal antibody A12, human monoclonal antibody B2, human monoclonal antibody E9, human monoclonal antibody 3D5, human monoclonal antibody 3D1, human monoclonal antibody 4D1, or human monoclonal antibody 3H9.

In some embodiments, the doppel-targeting moiety is an anti-doppel antibody having heavy chain constant domain sequence at least 85%, 90%, 95%, 99%, or 100% identical to the heavy chain constant domain sequence of human monoclonal antibody A12, human monoclonal antibody B2, human monoclonal antibody E9, human monoclonal antibody 3D5, human monoclonal antibody 3D1, human monoclonal antibody 4D1, or human monoclonal antibody 3H9.

In some embodiments, the doppel-targeting moiety is an anti-doppel antibody having light chain constant domain sequence at least 85%, 90%, 95%, 99%, or 100% identical to the light chain constant domain sequence of human monoclonal antibody A12, human monoclonal antibody B2, human monoclonal antibody E9, human monoclonal antibody 3D5, human monoclonal antibody 3D1, human monoclonal antibody 4D1, or human monoclonal antibody 3H9.

TABLE 1
Full Heavy Chain Sequences of Human Anti-Doppel mAbs

| Ab | Full Heavy Chain Sequence |
|---|---|
| A12 | EVQLVESGGGLVQPGGSLRLSCAASGFTFSSYAMSWVRQAPGKGLEWVSAIS |
| | SDGGSTYYADSVKGRFTISRDNSKNTLYLQMNSLRAEDTAVYYCAKRRLWS |
| | YSFDYWGQGTLVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNHKP |
| | SNTKVDKKVEPPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVL |
| | HQDWLNGKEYKCKVSNKALPAPIEKTISKAKGQPREPQVYTLPPSRDELTKN |
| | QVSLTCLVKGFYPSDIAVEWESNGQPENNYKTTPPVLDSDGSFFLYSKLTVD |
| | KSRWQQGNVFSCSVMHEALHNHYTQKSLSLSPGK (SEQ ID NO: 9) |
| B2 | EVQLVESGGGLVQPGGSLRLSCAASGFTFSDYAMSWVRQAPGKGLEWVSAI |
| | SSSGGSTYYADSVKGRFTISRDNSKNTLYLQMNSLRAEDTAVYYCAKLYYT |
| | YEVLDIWGQGTFVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNHKP |
| | SNTKVDKKVEPPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVL |
| | HQDWLNGKEYKCKVSNKALPAPIEKTISKAKGQPREPQVYTLPPSRDELTKN |
| | QVSLTCLVKGFYPSDIAVEWESNGQPENNYKTTPPVLDSDGSFFLYSKLTVD |
| | KSRWQQGNVFSCSVMHEALHNHYTQKSLSLSPGK (SEQ ID NO: 10) |
| E9 | QVQLVQSGAEVKKPGSSVKVSCKASGGTFSSYYMHWVRQAPGQGLEWMG |
| | RISPGGGGTIYAQKFQGRVTITADESTSTAYMELSSLRSEDTAVYYCARAQSE |
| | GLSYYFDVWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPGK (SEQ ID NO: 11) |
| 3D5 | EVQLVESGGGLVQPGGSLRLSCAASGFTFSDYAMSWVRQAPGKGLEWVSRI |
| | SSSGSSKYYADSVKGRFTISRDNSKNTLYLQMNSLRAEDTAVYYCAKWGSY |
| | GYGLVYYFDVWGQGTLVTVSSASTKGPSVFPLAPSSKSTSGGTAALGCLVKD |
| | YFPEPVTVSWNSGALTSGVHTFPAVLQSSGLYSLSSVVTVPSSSLGTQTYICN |
| | VNHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK (SEQ ID NO: 12) |
| 3D1 | EVQLVESGGGLVQPGGSLRLSCAASGFTFSDYAMSWVRQAPGKGLEWVSRI |
| | SGSGGTKYYADSVKGRFTISRDNSKNTLYLQMNSLRAEDTAVYYCAKHQVY |
| | WALDVWGQGTLVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQ |
| | VSLTCLVKGFYPSDIAVEWESNGQPENNYKTTPPVLDSDGSFFLYSKLTVDKS |
| | RWQQGNVFSCSVMHEALHNHYTQKSLSLSPGK (SEQ ID NO: 13) |
| 4D1 | EVQLVESGGGLVQPGGSLRLSCAASGFTFSDYAMSWVRQAPGKGLEWVSAI |
| | SSSGGEKYYADSVKGRFTISRDNSKNTLYLQMNSLRAEDTAVYYCAKHGYG |
| | QEYYYFDVWGQGTLVTVSSASTKGPSVFPLAPSSKSTSGGTAALGCLVKDYF |
| | PEPVTVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFLYSKLT |
| | VDKSRWQQGNVFSCSVMHEALHNHYTQKSLSLSPGK (SEQ ID NO: 14) |
| 3H9 | EVQLVESGGGLVQPGGSLRLSCAASGFTFSSYAMSWVRQAPGKGLEWVSGIS |
| | GSGSRTDYADSVKGRFTISRDNSKNTLYLQMNSLRAEDTAVYYCAKVYTYT |
| | RGFAFDLWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTV |
| | DKSRWQQGNVFSCSVMHEALHNHYTQKSLSLSPGK (SEQ ID NO: 15) |

TABLE 2
Heavy Chain CDR Sequences of Human Anti-Doppel mAbs
Heavy Chain CDR Sequence

| Antibody | CDRH1 | CDRH2 | CDRH3 |
|---|---|---|---|
| A12 | SYAMS | AISSDGGSTYYADSVKG | RRLWSYSFDY |
| | (SEQ ID NO: 16) | (SEQ ID NO: 23) | (SEQ ID NO: 30) |
| B2 | DYAMS | AISSSGGSTYYADSVKG | LYYTYEVLDI |
| | (SEQ ID NO: 17) | (SEQ ID NO: 24) | (SEQ ID NO: 31) |
| E9 | SYYMH | RISPGGGGTIYAQKFQG | AQSEGLSYYFDV |
| | (SEQ ID NO: 18) | (SEQ ID NO: 25) | (SEQ ID NO: 32) |
| 3D5 | DYAMS | RISSSGSSKYYADSVKG | WGSYGYGLVYYFDV |
| | (SEQ ID NO: 19) | (SEQ ID NO: 26) | (SEQ ID NO: 33) |
| 3D1 | DYAMS | RISGSGGTKYYADSVKG | HQVYWALDV |
| | (SEQ ID NO: 20) | (SEQ ID NO: 27) | (SEQ ID NO: 34) |
| 4D1 | DYAMS | AISSSGGEKYYADSVKG | HGYGQEYYYFDV |
| | (SEQ ID NO: 21) | (SEQ ID NO: 28) | (SEQ ID NO: 35) |
| 3H9 | SYAMS | GISGSGSRTDYADSVKG | VYTYTRGFAFDL |
| | (SEQ ID NO: 22) | (SEQ ID NO: 29) | (SEQ ID NO: 36) |

TABLE 3
Light Chain Sequences of Human Anti-Doppel mAbs

| | Light Chain |
|---|---|
| Ab | Variable Region Sequence or full Light Chain Sequence |
| A12 | DIQMTQSPSSLSASVGDRVTITCRASQSIGSYLNWYQQKPGKAPKLLIYAASTL |
| | QSGVPSRFSGSGSGTDFTLTISSLQPEDFATYYCQQSYSFPWTFGQGTKVEIKR |
| | TVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 37) |
| B2 | DIQMTQSPSSLSASVGDRVTITCRASQSISNYLNWYQQKPGKAPKLLIYAASRL |
| | QSGVPSRFSGSGSGTDFTLTISSLQPEDFATYYCQQSYSFPWTFGQGTKVEIKR |
| | TVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 38) |
| E9 | DIQMTQSPSSLSASVGDRVTITCRASQSIRNYLNWYQQKPGKAPKLLIYAASN |
| | LQSGVPSRFSGSGSGTDFTLTISSLQPEDFATYYCQQSSSFPLTFGQGTKVEIKR |
| | TVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 39) |
| 3D5 | DIQMTQSPSSLSASVGDRVTITCRASQSISSYLNWYQQKPGKAPKLLIYAASSL |
| | QSGVPSRFSGSGSGTDFTLTISSLQPEDFATYYCQQSESFPLTFGQGTKVEIKRT |
| | VAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 40) |
| 3D1 | DIQMTQSPSSLSASVGDRVTITCRASQSISNWLNWYQQKPGKAPKLLIYATSSL |
| | QSGVPSRFSGSGSGTDFTLTISSLQPEDFATYYCQQSYSYPWTFGQGTKVEIKR |
| | TVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 41) |
| 4D1 | DIQMTQSPSSLSASVGDRVTITCRASQSISNYLNWYQQKPGKAPKLLIYAASTL |
| | QSGVPSRFSGSGSGTDFTLTISSLQPEDFATYYCQQSYSTPWTFGQGTKVEIKR |
| | TVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 42) |
| 3H9 | DIQMTQSPSSLSASVGDRVTITCRASQSISNYLNWYQQKPGKAPKLLIYAASTL |
| | QSGVPSRFSGSGSGTDFTLTISSLQPEDFATYYCQQSYSFPYTFGQGTKVEIKRT |
| | VAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 43) |

TABLE 4
Light Chain CDR Sequences of Human Anti-Doppel mAbs
Light Chain CDR Sequence

| Antibody | CDRL1 | CDRL2 | CDRL3 |
|---|---|---|---|
| A12 | RASQSIGSYLN | AASTLQS | QQSYSFPWT |
| | (SEQ ID NO: 44) | (SEQ ID NO: 51) | (SEQ ID NO: 58) |
| B2 | RASQSISNYLN | AASRLQS | QQSYSFPWT |
| | (SEQ ID NO: 45) | (SEQ ID NO: 52) | (SEQ ID NO: 59) |
| E9 | RASQSIRNYLN (SEQ | AASNLQS | QQSSSFPLT |
| | ID NO: 46) | (SEQ ID NO: 53) | (SEQ ID NO: 60) |
| 3D5 | RASQSISSYLN | AASSLQS | QQSESFPLT |
| | (SEQ ID NO: 47) | (SEQ ID NO: 54) | (SEQ ID NO: 61) |
| 3D1 | RASQSISNWLN | ATSSLQS | QQSYSYPWT |
| | (SEQ ID NO: 48) | (SEQ ID NO: 55) | (SEQ ID NO: 62) |
| 4D1 | RASQSISNYLN | AASTLQS | QQSYSTPWT |
| | (SEQ ID NO: 49) | (SEQ ID NO: 56) | (SEQ ID NO: 63) |
| 3H9 | RASQSISNYLN | AASTLQS | QQSYSFPYT |
| | (SEQ ID NO: 50) | (SEQ ID NO: 57) | (SEQ ID NO: 64) |

As discussed above, due to the localization of doppel on tumor cells, an anti-doppel antibody suitable for use in the ADCs and methods described herein may be capable of targeting tumor cells, and thus exhibit one or more properties such as being capable of recognizing tumor cells, being capable of binding to tumor cells, being internalized into tumor cells, and exhibiting cytocidal activity against tumor cells, etc.

As also discussed above, due to the localization of doppel on tumor endothelial cells (TECs), an anti-doppel antibody suitable for use in the ADCs and methods described herein may be capable of targeting tumor endothelial cells (TECs), and thus exhibit one or more properties such as being capable of recognizing TECs, being capable of binding to TECs, being internalized into TECs, and exhibiting cytocidal activity against TECs, etc.

Because the ADC may include a separate chemotherapeutic agent, it is not essential that the doppel-targeting antibody itself exhibits an anti-tumor effect. Nevertheless, in some embodiments the doppel-targeting antibody itself exhibits an anti-tumor effect. As discussed above, the doppel-targeting antibody advantageous is internalized into tumor cells, to promote the cytotoxic effect of the chemotherapeutic compound in a manner that specifically and selectively targets tumor cells.

Binding activity of the antibody with tumor cells can be confirmed using flow cytometry. Internalization of the antibody into tumor cells can be confirmed using (1) an assay comprising visualizing antibody incorporated in cells under a fluorescence microscope using a secondary antibody (fluorescently labeled) bound to the doppel-targeting antibody (see, e.g., Adams et al., Cell Death and Differentiation (2008) 15, 751-761), (2) an assay comprising measuring the amount of fluorescence incorporated in cells using a secondary antibody (fluorescently labeled) bound to the doppel-targeting antibody (Austin et al., Mol. Biol. of the Cell, 2004, 15, 5268-5282), or (3) a Mab-ZAP assay using an immunotoxin bound to the doppel-targeting antibody wherein the toxin is released upon incorporation into cells to inhibit cell growth (see, e.g., Kohls et al., BioTechniques, 2000, 28, 162-165). For example, a recombinant complex protein of a diphtheria toxin catalytic domain and protein G may be used as the immunotoxin.

Additionally or alternatively, antibodies and antibody fragments can be screened for doppel-binding activity using conventional techniques. For example, measurement of absorbance, enzyme-linked immunosorbent assay (ELISA), enzyme immunoassay (EIA), radioimmunoassay (MA), western blot assay, and/or immunofluorescence may be used to measure doppel-binding activity. For example, for ELISA, a known anti-doppel antibody can be immobilized on a plate, doppel applied to the plate, and then a sample containing a test antibody, such as culture supernatant of antibody-producing cells or purified antibodies, can be applied. Then, a secondary antibody that recognizes the primary antibody and is labeled with an enzyme, such as alkaline phosphatase, is applied, and the plate is incubated. Next, after washing, an enzyme substrate, such as nitrophenyl phosphate, is added to the plate and the absorbance is measured to evaluate the antigen-binding activity of the sample. C-terminal or N-terminal fragment of Doppel protein may be used as an antigen. In another example, surface plasmon resonance analysis may be used to evaluate the activity of the antibody according to the present invention.

In some embodiments, the anti-doppel antibody binds one or more forms of doppel, such as one or more of the monomeric, dimeric, glycosylated, and non-glycosylated forms discussed above. In some embodiments, the anti-doppel antibody binds one or more forms of human Doppel, such as one or more of the monomeric, dimeric, glycosylated, and non-glycosylated forms discussed above. In some embodiments, the anti-doppel antibody binds one or more forms of a non-human species of doppel, such as one or more of the monomeric, dimeric, glycosylated, and non-glycosylated forms discussed above.

In some embodiments, the anti-doppel antibody preferentially binds to one or more of the forms of doppel described above, such as preferentially binding to one or more forms of doppel described above as compared to one or more of the other forms. In some embodiments, the anti-doppel antibody preferentially binds to one or more forms of human doppel. In some embodiments, the anti-doppel antibody preferentially binds to one or more forms of a non-human species of doppel.

In some embodiments, the anti-doppel antibody preferentially binds to one of the forms of doppel described above, such as preferentially binding to one of the forms of doppel described above as compared to the other forms. In some embodiments, the anti-doppel antibody preferentially binds to one form of human doppel. In some embodiments, the anti-doppel antibody preferentially binds to one form of a non-human species of doppel.

Antitumor activity of the antibody can be confirmed in vitro by determining inhibitory activity against cell growth. For example, a cancer cell line overexpressing a target protein for the antibody can be cultured, and the antibody added at varying concentrations into the culture system to determine inhibitory activity against focus formation, colony formation, and spheroid growth. Antitumor activity can be confirmed in vivo, for example, by administering the antibody to a nude mouse with a transplanted tumor cell line highly expressing the target protein and determining change in cancer (tumor) cells.

Doppel-targeting moieties suitable for use in the ADCs and methods described herein can be prepared using methodology known in the art. An anti-doppel antibody suitable for use in the ADCs and methods described herein can be derived from (e.g., raised in) any species. Examples of typical species include human, rat, mouse, and rabbit. An anti-doppel antibody derived from a non-human species may be chimeric or humanized. An anti-doppel antibody suitable for use in the ADCs and methods described herein can be a polyclonal antibody or a monoclonal antibody.

For example, antibodies can be raised in a host (such as a mammalian host) using an antigen comprising the doppel protein or a fragment thereof, such as an N-terminal or globular domain thereof, and screened for their ability to bind to doppel and, optionally, inhibit its interaction with a tyrosine kinase receptor (e.g., VEGFR2, etc.). For example, polyclonal antibodies against doppel may be prepared by collecting blood from a mammal immunized with doppel and having desired antibodies in the serum, and by separating the serum from the blood by methods known in the art. Serum containing polyclonal antibodies and/or fraction containing polyclonal antibodies may be isolated and purified.

Monoclonal antibodies for use in the ADCs and methods described herein can be produced by methods known to those skilled in the art. For example, immune cells may be collected from an antigen-immunized non-human mammal having desired antibodies in its serum and subjected to cell fusion. The immune cells used for cell fusion are typically obtained from the spleen. Other suitable parental cells that can be fused with the immunocyte include, for example, mammalian myeloma cells, such as mammalian myeloma cells having an acquired property for selection of fused cells by drugs. The immunocyte and myeloma cells can be fused according to known methods. See, e.g., Milstein et al., Methods Enzymol., 1981, 73, 3-46. Hybridomas obtained by the cell fusion may be selected by cultivating them in a standard selection medium, such as HAT medium (hypoxanthine, aminopterin, and thymidine-containing medium). The cell culture is typically grown in HAT medium for several days to several weeks, e.g., a time sufficient to allow all other cells except for the desired hybridoma (non-fused cells) to die. Then, a standard limiting dilution can be performed to screen and clone a hybridoma cell producing the desired antibody.

Humanized forms of non-human (e.g., murine) antibodies can be obtained as chimeric antibodies, which contain minimal sequences derived from non-human immunoglobulin. In general, a humanized antibody comprises at least one or two variable domains in which variable regions are derived from non-human immunoglobulin and framework regions (FR) correspond to a human immunoglobulin sequence. Thus, in some embodiments, the anti-doppel antibody used herein comprises a human antibody framework region. Such antibodies can be prepared by know techniques. A humanized antibody optionally may contain at least a portion of an immunoglobulin constant region (Fc), typically that of a human immunoglobulin. See, e.g., Jones et al., Nature 1986, 321, 522-525; Reichmann et al., Nature, 1988, 332, 323-329; Presta, Curr. Op. Struct. Biol., 1992, 2, 593-596.

As another method to obtain antibodies useful in the ADCs and methods described herein, transgenic animals with human antibody genes may be immunized with the doppel protein, doppel protein-expressing cells, or their lysates. The resulting antibody-producing cells can be collected and fused with myeloma cells to obtain a hybridoma, from which human antibodies against doppel can be prepared generally as outlined above. Alternatively, an immune cell, such as an immunized lymphocyte, producing antibodies may be immortalized by an oncogene and used for preparing monoclonal antibodies.

Monoclonal antibodies against doppel useful in the ADCs and methods described herein can be also prepared using recombinant genetic engineering techniques. See, e.g., Borrebaeck et al., THERAPEUTIC MONOCLONAL ANTIBODIES (MacMillan Publishers Ltd. (1990). For example, a DNA encoding an antibody against doppel may be cloned from an immune cell, such as a hybridoma or an immunized lymphocyte producing the antibody, inserted into an appropriate vector, and introduced into host cells to prepare a recombinant anti-doppel antibody.

As noted above, the doppel-targeting moiety of the ADCs described herein may be an antibody fragment that binds to doppel. As noted above, as used herein, the term “antibody fragment that binds to doppel” includes any doppel-binding fragment of an antibody or antibody-like molecule, including, but not limited to, Fab fragments, Fab′ fragments, F(ab′)2 fragments, Fv fragments, and smaller fragments, diabodies, etc. An antibody “fragment” may be prepared from a full-length antibody or may be synthesized as a “fragment” for example, using recombinant techniques. A doppel-binding antibody fragment may be generated by treating a doppel-binding antibody with an enzyme, such as papain or pepsin. Alternatively, a gene encoding a doppel antibody fragment may be constructed, inserted into an expression vector, and expressed in an appropriate host cell. See, e.g., Co et al., J. Immunol. 152: 2968-2976 (1994); Better and Horwitz, Methods Enzymol. 178:476-496 (1989); Pluckthun and Skerra, Methods Enzymol. 178:497-515 (1989); Lamoyi, Methods Enzymol. 121:652-663 (1986); Rousseaux et al., Methods Enzymol. 121:663-669 (1986); Bird and Walker, Trends Biotechnol. 9:132-137 (1991).

### Cleavable Linker

As noted above, the anti-doppel ADCs described herein include a cleavable linker. In some aspects, linkage of the anti-doppel moiety to the therapeutic agent drug via a cleavable linker may serve a dual purpose of linking the therapeutic agent to the anti-doppel moiety in an inactive form until it reaches the target site (therefore preventing systemic toxicities that may be associated with a chemotherapeutic agent, for example), and releasing the therapeutic agent at the target site (thereby maximizing efficacy at the desired site).

Suitable cleavable linkers for ADCs are known in the field, and include peptide linkers and non-peptide linkers. Typically, the cleavable linker of an anti-doppel ADC conjugate as described herein is cleavable under one or both of intracellular conditions and extracellular conditions. In some aspects, the linker is cleavable by an intracellular protease, such as lysosomal proteases or endosomal proteases. In some aspects, the linker is hydrolysable at a pH of less than 5.5. In some aspects, the linker is cleavable by a caspase, as discussed in more detail below.

In some aspects, the linker comprises or consists of a dipeptide linker. In some aspects, the dipeptide linker is a valine-citrulline (Val-Cit) linker, a valine-alanine (Val-Ala) linker, or a phenylalanine-lysine (Phe-Lys) linker, which are cleavable by cathepsin B.

In some aspects, the linker is or comprises a disulfide linker that is cleaved in a reduced environment.

In some aspects, the linker is or comprises a hydrazone linker that is hydrolyzable at a pH of less than 5.5.

As noted above, in some aspects, the linker is a caspase-cleavable peptide linker. Examples of suitable caspase-cleavable peptide linkers are described in U.S. Pat. No. 10,357,572, the entire contents of which are incorporated herein by reference.

As used herein, the term “caspase” refers to cysteine-aspartic proteases and cysteine-dependent aspartate-directed proteases that are activated (e.g., expressed) by cells undergoing apoptosis. In specific embodiments, the caspase is caspase-3, caspase-7, and/or caspase-9. Caspases are not specific to tumor cells per se, but are expressed as a result of cell apoptosis. Therefore, caspase has to be activated by another agent. This gives drugs which have caspase-cleavable peptide linkers (such as linkers comprising or consisting of the DEVD peptide sequence (SEQ ID NO: 4)) several unique advantages. First, caspase-cleavable peptide linkers are cleaved independently of tumor enzyme expression, which can be quite variable. Second, caspase-cleavable peptide linkers only are cleaved in the presence of caspase, e.g., in the presence of cells undergoing apoptosis. Furthermore, when the chemotherapeutic agent payload itself is non-selective, cleavage of the linker will release chemotherapeutic agent that can kill nearby tumor cells regardless of heterogeneity (this is known as the bystander killing effect). Even more notably, cleavage of a caspase-cleavable peptide linker can propagate cleavage of additional linkers, exerting an amplification effect. For example, cells that are killed by the chemotherapeutic agent will expresses caspase, which will in turn cleave caspase-cleavable peptide linkers of additional ADCs, which will release their chemotherapeutic agents, which will kill more tumor cells, and so on, effectively amplifying both the strength and duration of the response. (See, e.g., Sun et al., Bioconjugate Chem. 2020, 31, 4, 1012-1024.; Kovtun et al. Cancer Research (2006) 66 (6): 3214-21).

As used herein, the term “caspase-cleavable peptide linker” refers to a peptide sequence of two or more amino acid residues that is capable of being cleaved by caspase. In some aspects, the caspase cleavable peptide linker is cleavable by caspase-3 or caspase-7, such as peptides comprising the sequence Asp-Xaa-Xaa-Asp (where “Xaa” represents any amino acid, in L- or D-isomer form). In some aspects, the caspase-cleavable peptide linker is cleavable by caspase-9, such as peptides comprising the amino acid sequence Leu-Xaa-Xaa-Asp or Val-Xaa-Xaa-Asp (where “Xaa” represents any amino acid, in L- or D-isomer form).

In specific aspects, the caspase-cleavable peptide linker comprises or consists of one of the following sequences:

(SEQ ID NO: 4)
Asp-Glu-Val-Asp
(SEQ ID NO: 5)
Asp-Leu-Val-Asp
(SEQ ID NO: 6)
Asp-Glu-Ile-Asp,
or
(SEQ ID NO: 7)
Leu-Glu-His-Asp.

In specific aspects, the caspase-cleavable peptide linker comprises or consists of the sequence Lys-Gly-Asp-Glu-Val-Asp (SEQ ID NO:8), also denoted as KGDEVD (SEQ ID NO:8).

Generally speaking, the anti-doppel ADCs described herein are inactive until the linker is cleaved. Thus, the anti-doppel ADCs described herein comprising a chemotherapeutic agent exert minimal damage to healthy cells. Moreover, anti-doppel ADCs described herein that have a caspase-cleavable peptide linker only are activated in the presence of caspase, e.g., in the presence of cells undergoing apoptosis, such as tumor cells undergoing apoptosis. Thus, anti-doppel ADCs described herein that have a caspase-cleavable peptide linker are specific for their target via two modalities: (i) target specificity provided by the doppel-targeting moiety and (ii) target specificity provided by the caspase-cleavable peptide linker. As discussed above, anti-doppel ADCs described herein that have a caspase-cleavable peptide linker have an additional advantage due to the amplification effect. Still further, anti-doppel ADCs described herein that have a caspase-cleavable peptide linker wherein the four C-terminal amino acid residues of the caspase-cleavable peptide linker are selected from Asp-Xaa-Xaa-Asp, Leu-Xaa-Xaa-Asp, and Val-Xaa-Xaa-Asp, where Xaa represents any amino acid residue, have an additional advantage in that the linker is cleaved at its point of conjugation to the therapeutic agent, releasing the therapeutic agent in an unmodified form that will exhibit its intrinsic therapeutic effect (e.g., an antitumor or cyctotoxic effect).

TABLE 5
Caspase-Cleavable Peptide Linkers
Caspase-Cleavable Peptide Linker

| | |
|---|---|
| (Xaa represents any | Asp-Xaa-Xaa-Asp |
| amino acid residue) | |
| (Xaa represents any | Leu-Xaa-Xaa-Asp |
| amino acid residue) | |
| (Xaa represents any | Val-Xaa-Xaa-Asp |
| amino acid residue) | |
| SEQ ID NO: 4 | Asp-Glu-Val-Asp |
| SEQ ID NO: 5 | Asp-Leu-Val-Asp |
| SEQ ID NO: 6 | Asp-Glu-Ile-Asp |
| SEQ ID NO: 7 | Leu-Glu-His-Asp |
| SEQ ID NO: 8 | Lys-Gly-Asp-Glu-Val-Asp |

### Therapeutic Agent

As noted above, the anti-doppel ADCs described herein include a therapeutic agent.

In some aspects, the therapeutic agent is or comprises a chemotherapeutic agent. The terms “chemotherapeutic agent” and “cytotoxic agent” generally are used interchangeably herein. As used herein, the term “chemotherapeutic agent” refers to a moiety useful to treat cancer, such as a small molecule chemical compound used to treat cancer. Similarly, as used herein, the term “cytotoxic agent” refers to a moiety useful to induce cell death (apoptosis), such as a small molecule chemical compound used to induce cell death in target cells. In specific embodiments, the chemotherapeutic agent induces apoptosis in target cells, e.g., in tumor cells and tumor tissue. Any chemotherapeutic agent known in the art can be used as a chemotherapeutic agent in the anti-doppel ADCs described herein. In general, a chemotherapeutic agent is used that has, or can be prepared to have, a substituent, structure, or moiety that can be conjugated to another moiety of the ADC (directly or through a linkage). Suitable chemotherapeutic agents include the cytotoxic agents described in U.S. Pat. No. 10,357,572, the entire contents of which are incorporated herein by reference.

Suitable cytotoxic agents include, for example, an auristatin, a DNA minor groove binding agent, a DNA minor groove alkylating agent, an enediyne, a lexitropsin, a duocarmycin, a taxane, a puromycin, a dolastatin, a maytansinoid, and a vinca alkaloid. In some aspects, the cytotoxic agent is selected from auristatin phenylalanine phenylenediamine (AFP), monomethyl auristatin F (MMAF), monomethyl auristatin E (MMAE), auristatin E, paclitaxel, docetaxel, CC-1065, SN-38, topotecan, morpholino-doxorubicin, rhizoxin, cyanomorpholino-doxorubicin, dolastatin-10, echinomycin, combretatstatin, calicheamicin, maytansine, DM1, netropsin, and derivatives thereof. Other suitable cytotoxic agents include anti-tubulin agents, such as an auristatin, a vinca alkaloid, a podophyllotoxin, a taxane, a baccatin derivative, a cryptophysin, a maytansinoid, a combretastatin, and a dolastatin. In specific aspects, the anti-tubulin agent is auristatin phenylalanine phenylenediamine (AFP), monomethyl auristatin F (MMAF), monomethyl auristatin E (MMAE), auristatin E, Vincristine, vinblastine, vindesine, vinorelbine, VP-16, camptothecin, paclitaxel, docetaxel, epothilone A, epothilone B, nocodazole, colchicines, colcimid, estramustine, cemadotin, discodermolide, maytansine, DM1, or eleutherobin. In some embodiments, the anti-tubulin is monomethyl auristatin E (MMAE).

In some embodiments, the chemotherapeutic agent is an anthracycline, such as doxorubicin, daunorubicin, epirubicin, idarubicin, valrubicin, or a derivative thereof; an antibiotic, such as actinomycin-D, bleomycin, mitomycin-C, calicheamicin, or a derivative thereof; an alkylating agent, such as cyclophosphamide, mechlorethamine, uramustine, melphalan, chlorambucil, ifosfamide, bendamustine, carmustine, lomustine, streptozocin, busulfan, dacarbazine, temozolomide, thiotepa, altretamine, duocarmycin, or a derivative thereof; a platinum-based agent, such as cisplatin, carboplatin, nedaplatin, oxaliplatin, satraplatin, triplatin tetranitrate, or a derivative thereof; an antimetabolite, such as 5-fluorouracil, 6-mercaptopurine, capecitabine, cladribine, clofarabine, cystarbine, floxuridine, fludarabine, gemcitabine, hydroxyurea, methotrexate, pemetrexed, pentostatin, thioguanine, or a derivative thereof; a topoisomerase inhibitor, such as exatecan, camptothecin, topotecan, irinotecan, etoposide, teniposide, mitoxantrone, or a derivative thereof; a mitotic inhibitor, such as paclitaxel, docetaxel, izabepilone, vinblastine, vincristine, vindesine, vinorelbine, estramustine, maytansine, DM1 (mertansine), DM4, dolastatin, auristatin E, auristatin F, monomethyl auristatin E, monomethyl auristatin F, or a derivative thereof. In specific embodiments, the chemotherapeutic agent is doxorubicin. In specific embodiments, the chemotherapeutic agent is danorubicin. In specific embodiments, the chemotherapeutic agent is exatecan.

In some embodiments, the chemotherapeutic agent is selected from 5-FU, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, exatecan, camptothecin, carboplatin, 10-hydroxycamptothecin, carmustine, celebrex, chlorambucil, cisplatin, COX-2 inhibitors, irinotecan, SN-38, cladribine, crizotinib, cyclophosphamide, cytarabine, dacarbazine, dasatinib, dinaciclib, docetaxel, dactinomycin, daunorubicin, doxorubicin, epidophyllotoxin, erlotinib, entinostat, estrogen receptor binding agents, etoposide, exemestane, fingolimod, floxuridine, fludarabine, flutamide, flavopiridol, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechloresthamine, melphalan, mercaptopurine, methotrexate, mitoxantrone, mithramycin, mitomycin, mitotane, navelbine, neratinib, nilotinib, nitrosurea, olaparib, plicomycin, procarbazine, paclitaxel, PCI-32765, pentostatin, PSI-341, raloxifene, semustine, sorafenib, streptozocin, SU11248, sunitinib, tamoxifen, temazolomide, transplatin, thalidomide, thioguanine, thiotepa, teniposide, topotecan, uracil mustard, vatalanib, vinorelbine, vinblastine, vincristine, vinca alkaloids and ZD183.

As noted above, a chemotherapeutic agent useful in the anti-doppel ADCs described herein include monomethyl auristatin E (MMAE), which has the chemical name:
((S)—N-((3R,4 S, 5 S)-14(S)-2-41R,2R)-3-(((1 S,2R)-1-hydroxy-1-phenylpropan-2-yl)amino)-1-m ethoxy-2-methyl-3-oxopropyl)pyrrolidin-1-yl)-3-m ethoxy-5-methyl-1-oxoheptan-4-yl)-N,3-dim ethyl-2-((S)-3-methyl-2-(methylamino)butanamido)butanamide
and the following chemical formula:

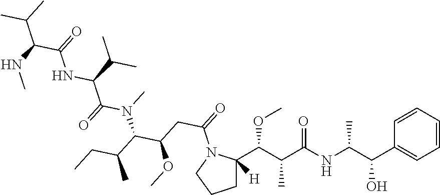 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

As noted above, a chemotherapeutic agent useful in the anti-doppel ADCs described herein includes exatecan which has the chemical name:
(1 S,9 S)-1-Amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione
and the following chemical formula:

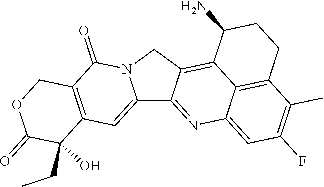 [SMILES: CC[C@@]1(O)C(=O)OCc2c1cc1n(c2=O)Cc2c-1nc1cc(F)c(C)c3c1c2[C@@H](N)CC3]

As noted above, other suitable chemotherapeutic agents include doxorubicin, daunorubicin, mitomycin C, bleomycin, cyclocytidine, vincristine, vinblastine, methotrexate, platinum-based antitumor agent (cisplatin or derivatives thereof), taxol and derivatives thereof, and camptothecin and derivatives thereof, including exatecan.

In some aspects, the therapeutic agent is or comprises an immunomodulatory agent. As used herein, the term “immunomodulatory agent” refers to agents that can help the body defend against pathogens, e.g., tumor cells, by adjusting the immune response. In specific embodiment, the immunomodulator is selected from cytokines, lymphokines, monokines, stem cell growth factors, lymphotoxins, hematopoietic factors, colony stimulating factors (CSF), interrerons (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, transforming growth factor (TGF), TGF-alpha, TGF-beta, insulin-like growth factor (IGF), erythropoietin, thrombopoietin, tumor necrosis factor (TNF), TNF-alpha, TNF-beta, mullerian-inhibiting substance, mouse gonadotropin-associated peptide, inhibin, activin, vascular endothelial growth factor, integrin, interleukin (IL), granulocyte-colony stimulating factor (G-CSF), granulocyte macrophage-colony stimulating factor (GM-CSF), interferon-alpha, interferon-beta, interferon-gamma, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-23, IL-25, LIF, kit-ligand, FLT-3, angiostatin, thrombospondin, endostatin, toll-like receptor (TLR) agonists (such as CU-T12-9, Pam3CSK4, FSL-1, Pam2CSK4, and CL429), Poly(A:U), Poly(I:C), lipopolysaccharides (LPS), MPLA-SM, CRX-527, flagellin, thiazoquinoline derivatives, imidazoquinoline derivatives (such as CL097, gardiquimod, imiquimod, and resiquimod), adenine analogs, guanosine analogs, thymidine analogs, benzoazepine analogs, and CpG oligodeoxynucleotides (ODN) (such as ODN 1585, ODN 2216, ODN 2336, ODN 1668, ODN 1826, ODN 2006, ODN 2007, ODN BW006, ODN D-SL01, ODN 2395, ODN M362, and ODN D-SL03).

In some aspects, the therapeutic agent is or comprises a toxin. As used herein, the term “toxin” refers to naturally occurring organic elements produced by metabolic activities of living cells or organisms. In specific embodiment, then toxin is selected from ricin, abrin, ribonuclease (RNase), DNase 1, Staphylococcal enterotoxin-A, pokeweed antiviral protein, gelonin, diphtheria toxin, Pseudomonas exotoxin, and Pseudomonas endotoxin.

In some aspects, the therapeutic agent is or comprises a radionuclide. The terms “radionuclide,” “radioactive nuclide,” “radioisotope,” or “radioactive isotope” generally are used interchangeably herein. As used herein, the term “radionuclide” refers to a nuclide that has excess nuclear energy making it unstable. Radionuclides occur naturally or are artificially produced. Expose to radionuclides can have harmful effects on living organisms. In specific embodiments, the radionuclide is selected from 11C, 13N, 15O, 32P, 33P, 47Sc, 51Cr, 57Co, 58Co, 59Fe, 62Cu, 67Cu, 67Ga, 75Br, 75Se, 76Br, 77As, 77Br, 80mBr, 89Sr, 90Y, 95Ru, 97Ru, 99Mo, 99mTc, 103mRh, 103Ru, 105Rh, 105Ru, 107Hg, 109Pd, 109Pt, 111Ag, 111In, 113mIn, 119Sb, 121mTe, 122mTe, 125I, 125mTe, 126I, 131I, 133I, 142Pr, 143Pr, 149Pm, 152Dy, 153Sm, 161Ho, 161Tb, 165Tm, 166Dy, 166Ho, 167Tm, 168TM, 169Er, 169Yb, 177Lu, 186Re, 188Re, 189mOs, 189Re, 198Ir, 194Ir, 197Pt, 198Au, 199Au, 203Hg, 211At, 211Bi, 211Pb, 212Bi, 212Pb, 213Bi, 215Po, 217At, 219Rn, 221Fr, 223Ra, 225Ac, 227Th and 255Fm.

In some embodiments, the therapeutic agent is or comprises a DNA cross-linking agent. In some embodiments, the DNA cross-linking agent is selected from indolionobenzodiazepine dimer (IGN), Pyrrolobenzodiazepine (PBD) and derivatives thereof.

### Linkages

In some embodiments of the anti-doppel ADCs described herein, the doppel-targeting moiety is joined directly or through a linker to the cleavable linker, and the cleavable linker may be joined directly or through a linker to the therapeutic agent.

Thus, in certain embodiments the doppel-targeting moiety is conjugated directly to, e.g., the caspase-cleavable peptide linker, such as by a covalent bond between a moiety on the doppel-targeting moiety and a moiety at the N-terminus of the peptide linker or on a side chain of the peptide linker. Independently, in some embodiments the cleavable linker (such as the caspase-cleavable peptide linker) is conjugated directly to the therapeutic agent, such as by a covalent bond between a moiety at the C-terminus of the peptide linker or on a side chain of the peptide linker and a moiety on the therapeutic agent.

Alternatively, one or both of the linkages may be through a linker. Any linker suitable for use in pharmaceutical compounds may be used for this purpose. Suitable linkers include p-aminobezylcarbamate (PABC).

Although the above description assumes that the doppel-targeting moiety is linked to the N-terminus of the caspase-cleavable peptide linker, while the therapeutic agent is linked to the C-terminus of the caspase-cleavable peptide linker, also contemplated are ADCs wherein the therapeutic agent is linked to the N-terminus of the caspase-cleavable peptide linker, while the doppel-targeting moiety is linked to the C-terminus of the caspase-cleavable peptide linker.

In still other embodiments, an anti-doppel ADC as described herein includes a caspase-cleavable peptide joined directly or through a linker to a therapeutic agent, which is joined directly or through a linker to a doppel-targeting moiety. For example, daunorubicin exhibits its chemotherapeutic effect when it is conjugated at its 14-CH₃ position to another moiety. Thus, caspase-induced cleavage need not release free daunorubicin in order to provide a chemotherapeutic effect. Thus, in some embodiments an ADC comprises a caspase-cleavable peptide joined directly or through a linker to daunorubicin, which is joined at its 14-CH₃ position directly or through a linker to a doppel-targeting moiety.

### Anti-Doppel ADCs

The anti-doppel ADCs described herein can be made by methods known in the art for preparing conjugates.

The anti-doppel ADCs described herein can be made in a step wise approach, such as by first making a cleavable linker-therapeutic agent conjugate, and then conjugating one or more cleavable linker-therapeutic agent conjugates to a doppel-targeting moiety. For example, a caspase cleavable peptide linker-therapeutic agent conjugate can be prepared as described in U.S. Pat. No. 10,357,572 (the entire contents of which are incorporated herein by reference), and then one or more thereof can be conjugated to a doppel-targeting moiety as described herein. Other suitable methodologies for preparing ADCs as described herein are known in the field and illustrated in the examples below.

The number of therapeutic agent drug molecules per doppel-targeting moiety (e.g., per doppel-targeting antibody) may impact the efficacy and safety of the ADC. Thus, the ADC may be prepared under reaction conditions that control the number of therapeutic agent drug molecules per doppel-targeting moiety, such as the relative amounts of reactants used, other reagents used, and other reaction conditions. Still, a mixture of ADCs having different numbers of conjugated therapeutic agent drug molecules may be obtained. Thus, the number of therapeutic agent drug molecules conjugated to an ADC may be specified by an average value representing the average number of conjugated therapeutic agent drug molecules per doppel-targeting moiety. Thus, in the examples that follow, unless otherwise specified (such as in the context of reporting the number of conjugated therapeutic agent drug molecules per doppel-targeting moiety of a given ADC in a mixture), the number of conjugated therapeutic agent drug molecules per doppel-targeting moiety refers to an average value.

Specific examples of anti-doppel ADCs are set forth in the examples below and in the figures, including 3D1-KGDEVD-MMAE (name=SEQ ID NO:67; structure=SEQ ID NO:68), 3D5-KGDEVD-MMAE (name=SEQ ID NO:69; structure=SEQ ID NO:70), 3H9-KGDEVD-MMAE (name=SEQ ID NO:65; structure=SEQ ID NO:66), 3H9-KGDEVD-Exatecan (name=SEQ ID NO:75; structure=SEQ ID NO:76), 4D1-KGDEVD-MMAE (name=SEQ ID NO:71; structure=SEQ ID NO:72), 3D1-vc-MMAE, 3D5-vc-MMAE, 3H9-vc-MMAE, and 4D1-vc-MMAE.

### Pharmaceutical Compositions

In some aspects, the anti-doppel ADC is provided in a pharmaceutical composition, such as a composition comprising the anti-doppel ADC and a pharmaceutically acceptable carrier, excipient, and/or diluent. Examples of suitable carriers, excipients, and diluents include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, minerals, and the like.

The pharmaceutical composition may be prepared for any route of administration, including any parenteral or local route of administration. In some aspects, the pharmaceutical composition is suitable for injection or infusion, such as for intravenous injection or infusion, such as being prepared as a sterile composition for injection or infusion. Appropriate components and excipients for such compositions are known in the art.

Examples of other formulations for parenteral administration include sterilized aqueous solutions, water-insoluble solutions, suspensions, emulsions, lyophilized formulations, and suppositories. Non-aqueous solutions and suspensions may include, for example, propylene glycol, polyethylene glycol, a plant oil such as olive oil, or injectable ester such as ethyloleate. A base for a suppository formulation may include, for example, witepsol, macrogol, Tween 61, cacao butter, laurin butter, glycerogelatin or the like.

In specific embodiments, the conjugate is dissolved in water or another pharmaceutically acceptable aqueous carrier in which the conjugate exhibits good solubility, optionally with or without other pharmaceutical acceptable excipients, preservatives, and the like.

Also provided are kits. A kit may comprise one or more of the ADCs as disclosed herein, contained in a suitable container, optionally together with instructions for use in a method as disclosed herein.

### Methods Using Anti-Doppel ADCs

As noted above, the anti-doppel ADCs described herein are useful in methods of treating doppel-associated diseases and conditions, including cancer.

The anti-doppel ADCs may be administered by any route of administration. In some aspects, the ADCs are administered intravenously. The dose of ADC administered will vary depending on the subject and the condition for which it is administered, and can be determined by someone of skill in the art. In some embodiments, the dose administered to a subject may be between about 1 mg/kg and about 100 mg/kg, including from about 5 mg/kg to about 75 mg/kg, such as from about 10 mg/kg to about 50 mg/kg, or greater. As discussed above, ADC's comprising chemotherapeutic agents typically exhibits lower toxicity than the chemotherapeutic agent alone, such that the dose of ADC administered may be higher than that which would be non-toxic for the chemotherapeutic agent alone.

As discussed above, embodiments wherein the ADC comprises a caspase-cleavable peptide linker and a chemotherapeutic agent may benefit from an amplification effect: Apoptosis is induced by an apoptosis inducing treatment, as disclosed above, resulting in expression of caspase. The ADC is administered, and the caspase-cleavable peptide linker is cleaved by the caspase, releasing the chemotherapeutic agent. The chemotherapeutic agent induces apoptosis of additional cells, resulting in additional expression of caspase, resulting in the caspase-induced cleavage/activation of additional ADC, resulting in amplified apoptosis. This amplification yields methods with high efficiency and specificity in killing target cells, such as target tumor cells. Moreover, this amplification effect can prolong the time interval between apoptosis inducing treatments and/or between administrations of doses of the ADC. Thus, in some embodiments, this amplification effect may reduce the amount of chemotherapeutic agent needed to treat a certain number of cancer cells.

As noted above, the ADC is inactive prior to cleavage of the caspase-cleavable peptide linker. Thus, the ADC is not toxic (or apoptotic) to healthy cells. In specific embodiments, the methods described herein reduce damage to normal cells by about 10%, 20%, 30%, 40%, 50%, 60%, 70%, 80% or more, as compared with administration of the same chemotherapeutic agent in non-conjugated form.

Moreover, the apoptotic effect of the ADC is selective to cells expressing caspase, e.g., cells undergoing apoptosis. Thus, once apoptosis is induced in a region of target cells (e.g., in target tissue), the methods described herein selectively and effectively induce apoptosis of other target cells, thereby, for example, treating cancer.

### EXAMPLES

### Example 1—Production of ADC Named 3H9-KGDEVD-MMAE (Name=SEQ ID NO:65)

The cytotoxic drug monomethyl auristatin E (MMAE) was linked to the anti-doppel human mAb 3H9 by the caspase-cleavable peptide linker consisting of Lys-Gly-Asp-Glu-Val-Asp (SEQ ID NO: 8) to give the ADC named 3H9-KGDEVD-MMAE (name=SEQ ID NO:65):

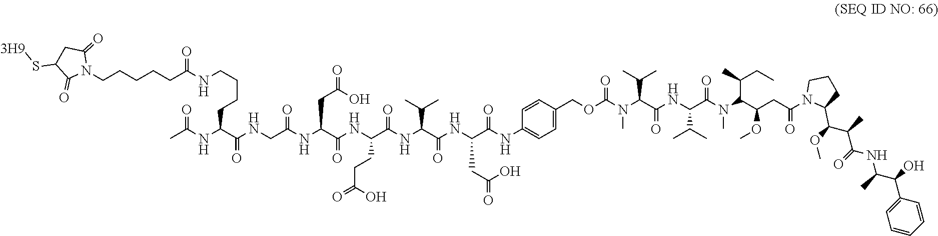

The synthesis of 3H9-KGDEVD-MMAE (SEQ ID NO:65), the general structure of which is depicted above (SEQ ID NO:66), is described below.

### A. Maleimide-KGDEVD-WAE (SEQ ID NO: 77) Drug-Linker Conjugate Synthesis

A maleimide-KGDEVD-MMAE (SEQ ID NO:77) conjugate is prepared as described in U.S. Pat. No. 10,357,572 (the entire contents of which are incorporated herein by reference). In particular, ε-maleimidocaproylate and MMAE are bound to the amino group of the Lys side chain and the C-terminus of an AcKGDEVD peptide (SEQ ID NO:1), respectively.

### B. Conjugation With mAb 3H9

To prepare the ADC, mAb 3H9 is partially reduced with tris(2-carboxyethyl)phosphine (TCEP) followed by reaction of reduced Cys residues with the maleimide-KGDEVD-MMAE (SEQ ID NO:77) conjugate (e.g., maleimide-terminated linker-payload).

In particular, 3H9 mAb is partially reduced via addition of 4.2 molar excess of tris(2-carboxyethyl)phosphine (TCEP) in 100 mM HEPES (4-(2-hydroxyethyl)-1-piperazineethanesulfonic acid buffer), pH 7.0, and 1 mM diethylenetriamine pentaacetic acid (DTPA) for 1 hour at 37° C. The thiol/Ab value is found by determining the reduced antibody concentration from the solution's BCA assay, and determining the thiol concentration by reaction with DTNB and determination of absorbance at 412 nm.

The reduced mAb is chilled on ice. The maleimide-terminated linker-payload is added to the reaction mixture at a linker-payload/mAb-thiol molar ratio of 2:1, and reacted for an additional 1 hour at 4° C. After the 1 hour incubation period, the reaction mixture is concentrated by centrifugal ultrafiltration and purified by elution through de-salting G25 in PBS at 4° C. The ADC 3H9-KGDEVD-MMAE (name=SEQ ID NO:65; structure=SEQ ID NO:66) is then filtered through a 0.2 micron filter under sterile conditions and immediately frozen at −80° C. The ADC is analyzed for (1) concentration, by BCA protein assay; (2) aggregation, by size exclusion chromatography; (3) residual free drug, by reverse phase HPLC; and (4) drug-antibody ratio (DAR), by hydrophobic interaction chromatography.

### Example 2—Production of ADC Named 3H9-Vc-MMAE

An ADC was prepared comprising MMAE as the cytotoxic drug, mAb 3H9 as the doppel-targeting moiety, and a cathepsin B-cleavable peptide linker consisting of Val-Cit to give the ADC named 3H9-vc-MMAE:

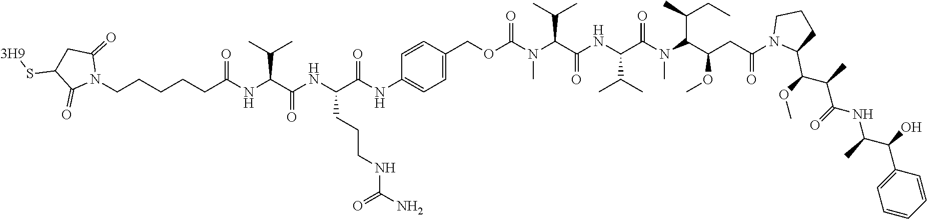

The synthesis of the ADC 3H9-vc-MMAE, the general structure of which is depicted above, is described below.

To prepare the ADC, mAb 3H9 is partially reduced with tris(2-carboxyethyl)phosphine (TCEP) followed by reaction of reduced Cys residues with the maleimide-terminated linker-payload (maleimidocapronic-valine-citruline-p-aminobenzyloxycarbonyl-MMAE, also known as mc-Val-Cit-PABA-MMAE, which can be purchased from MedChemExpress).

In particular, 3H9 mAb is partially reduced via addition of 4.2 molar excess of tris(2-carboxyethyl)phosphine (TCEP) in 100 mM HEPES (4-(2-hydroxyethyl)-1-piperazineethanesulfonic acid buffer), pH 7.0, and 1 mM diethylenetriamine pentaacetic acid (DTPA) for 1 hour at 37° C. The thiol/Ab value is found by determining the reduced antibody concentration from the solution's BCA assay, and determining the thiol concentration by reaction with DTNB and determination of absorbance at 412 nm.

The reduced mAb is chilled on ice. The maleimide-terminated linker-payload (mc-Val-Cit-PABA-MMAE, MedChemExpress) is added to the reaction mixture at a linker-payload/mAb-thiol molar ratio of 2:1, and reacted for an additional 1 hour at 4° C. After the 1 hour incubation period, the reaction mixture is concentrated by centrifugal ultrafiltration and purified by elution through de-salting G25 in PBS at 4° C. 3H9-vc-MMAE is then filtered through a 0.2 micron filter under sterile conditions and immediately frozen at −80° C. The ADC (3H9-vc-MMAE) is analyzed for (1) concentration, by BCA protein assay; (2) aggregation, by size exclusion chromatography; (3) residual free drug, by reverse phase HPLC; and (4) drug-antibody ratio (DAR), by hydrophobic interaction chromatography.

### Example 3—Production of ADC Named 3H9-KGDEVD-Exatecan (Name=SEQ ID NO:75)

The cytotoxic drug exatecan was linked to the anti-doppel human mAb 3H9 by the caspase-cleavable peptide linker consisting of Lys-Gly-Asp-Glu-Val-Asp (SEQ ID NO:8) to give the ADC named 3H9-KGDEVD-Exatecan (name=SEQ ID NO:75):

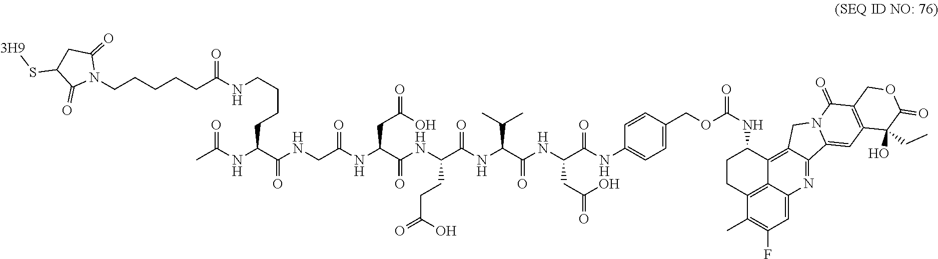

The synthesis of 3H9-KGDEVD-Exatecan (name=SEQ ID NO:75), the general structure of which is depicted above (SEQ ID NO:76), is described below.

### A. Maleimide-KGDEVD-Exatecan (SEQ ID NO:2) Drug-Linker Conjugate Synthesis

A maleimide-KGDEVD-Exatecan (SEQ ID NO:2) conjugate is prepared as described in U.S. Pat. No. 10,357,572 (the entire contents of which are incorporated herein by reference). In particular, ε-maleimidocaproylate and exatecan are bound to the amino group of the Lys side chain and the C-terminus of an AcKGDEVD peptide (SEQ ID NO:1), respectively.

### B. Conjugation with mAb 3H9

To prepare the ADC, mAb 3H9 is partially reduced with tris(2-carboxyethyl)phosphine (TCEP) followed by reaction of reduced Cys residues with the maleimide-KGDEVD-Exatecan (SEQ ID NO:2) conjugate (e.g., maleimide-terminated linker-payload).

In particular, 3H9 mAb is partially reduced via addition of 4.2 molar excess of tris(2-carboxyethyl)phosphine (TCEP) in 100 mM HEPES (4-(2-hydroxyethyl)-1-piperazineethanesulfonic acid buffer), pH 7.0, and 1 mM diethylenetriamine pentaacetic acid (DTPA) for 1 hour at 37° C. The thiol/Ab value is found by determining the reduced antibody concentration from the solution's BCA assay, and determining the thiol concentration by reaction with DTNB and determination of absorbance at 412 nm.

The reduced mAb is chilled on ice. The maleimide-terminated linker-payload is added to the reaction mixture at a linker-payload/mAb-thiol molar ratio of 2:1, and reacted for an additional 1 hour at 4° C. After the 1 hour incubation period, the reaction mixture is concentrated by centrifugal ultrafiltration and purified by elution through de-salting G25 in PBS at 4° C. The ADC 3H9-KGDEVD-Exatecan (name=SEQ ID NO:75; structure=SEQ ID NO:76) is then filtered through a 0.2 micron filter under sterile conditions and immediately frozen at −80° C. The ADC is analyzed for (1) concentration, by BCA protein assay; (2) aggregation, by size exclusion chromatography; (3) residual free drug, by reverse phase HPLC; and (4) drug-antibody ratio (DAR), by hydrophobic interaction chromatography.

### Example 4—Cytotoxicity Assay

The ability of anti-doppel ADCs as described herein to deliver a potent cytotoxic drug and eliminate doppel-expressing cells was assessed.

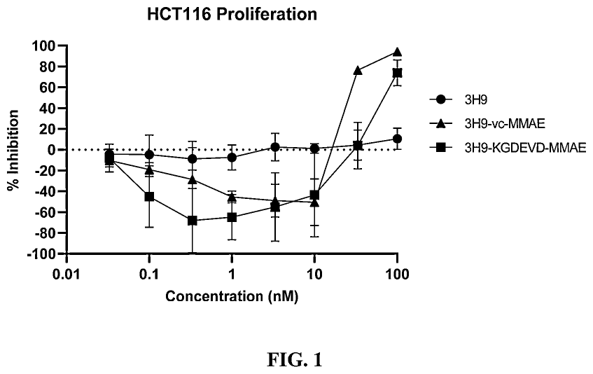

A cell line expressing doppel, HCT116, was selected and cultured with increasing concentrations of ADC. After 72 hours, viability of each culture was assessed. IC₅₀ values were calculated by logistic non-linear regression and reported as nM. The ADCs 3H9-KGDEVD-MMAE (name=SEQ ID NO:65; structure—SEQ ID NO:66) and 3H9-vc-MMAE prepared as described above inhibited growth of the doppel expressing cell line (HCT116), while 3H9 mAb alone did not. (FIG. 1)

TABLE 6
IC₅₀ values of anti-doppel ADCs on HCT116 cells.

| ADC | IC₅₀ (nM) |
|---|---|
| 3H9-KGDEVD-MMAE | 39.32 |
| (name = SEQ ID NO: 65; | |
| structure = SEQ ID NO: 66) | |
| 3H9-vc-MMAE | 28.65 |

### Example 5—Internalization of Anti-Doppel Ab by Doppel-Expressing Cell Lines

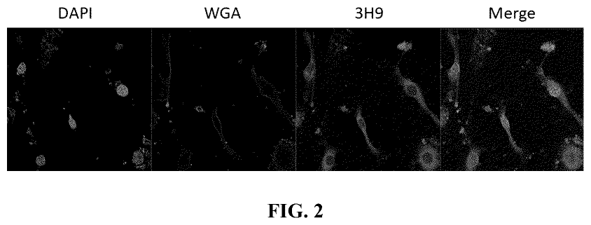

Immunofluorescence microscopy was conducted to study internalization of anti-doppel mAb into human colorectal tumor endothelial cells (HCTECs). A total of 1×10 4 HCTEC cells cultured in coverslips were treated with 0.1 mg/ml of 3H9 mAb conjugated with FITC. After incubating for 3 hours, the cells were washed with PBS and fixed in 10% neutral buffered formalin solution. Then, the cells were stained with 5 ug/ml WGA-Texas red solution overnight at 4° C. Subsequently, cells were washed with PBS, in situ mounting media with DAPI was added, and the cells were observed in a microscope. The results show that anti-doppel mAb 3H9 was internalized into doppel-expressing HCTEC cells. (FIG. 2)

### Example 6—Lysosomal Colocalization of Anti-Doppel Ab on Doppel-Expressing Cell Lines

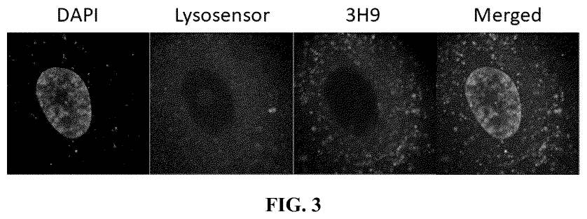

Immunofluorescence microscopy was conducted to study the lysosomal colocalization of anti-doppel mAb to human colorectal tumor endothelial cells (HCTECs). A total of 1×10⁴ HCTEC cells cultured in coverslips were treated with 0.1 mg/ml of 3H9 mAb conjugated with Cy5.5. After incubating for 3 hours, the cells were washed with PBS and treated with lysosensor for 2 hours at 37° C. Then, the cells were washed again with PBS and fixed in 10% neutral buffered formalin solution. The cells were washed with PBS, in situ mounting media with DAPI was added, and the cells were observed in a microscope. The results show that anti-doppel mAb 3H9 was colocalized with lysosomal marker in doppel-expressing HCTEC cells. (FIG. 3)

### Example 7—In Vivo Efficacy of Anti-Doppel ADCs in a Xenograft Model

To assess the anti-tumor efficacy of anti-doppel ADCs, an HCT116 xenograft tumor model was used. HCT116 cells (1×10\hat{ }⁷ cells/mouse) were inoculated into the left flank of balb-c/nu mice. When the average tumor volume reach 70-100 mm³, ADC was administered intravenously every 4 days for a total of 4 times. Tumor volume was measured every 4 days until the end of the experiments.

The ADCs assessed were 3D1-vc-MMAE, 3D5-vc-MMAE, 3H9-vc-MMAE and 4D1-vc-MMAE. ADCs 3D1-vc-MMAE, 3D5-vc-MMAE, and 4D1-vc-MMAE were prepared as described above for ADC 3H9-vc-MMAE, but using different anti-doppel antibodies (mAb 3D1, mAb 3D5, and mAb 4D1, the sequences of each of which are set forth above; these mAbs also are described in U.S. patent application Ser. No. 17/350,763 (the entire contents of which are incorporated herein by reference).

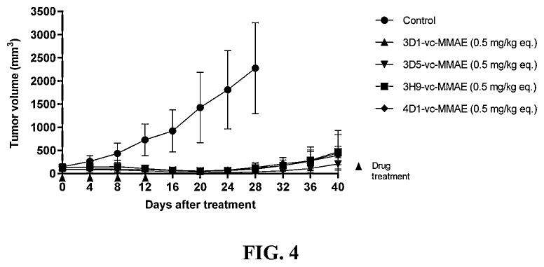

The results show that treatment with each ADC that was tested effectively suppressed the tumor growth. (FIG. 4)

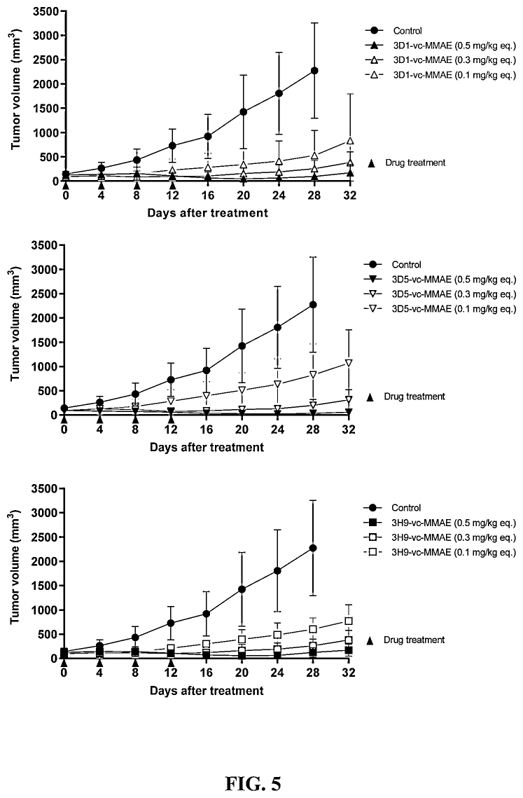

A dose-dependency study using the same tumor model and procedure was carried out using the ADCs 3D1-vc-MMAE, 3D5-vc-MMAE, and 3H9-vc-MMAE. The results show a dose-dependent effect of each ADC tested. (FIG. 5)

An in vivo efficacy test comparing ADCs prepared with different linkers was also carried out. The ADCs used were 3H9-KGDEVD-MMAE (name=SEQ ID NO: 65; structure—SEQ ID NO:66), 3H9-DEVD-MMAE (name=SEQ ID NO:73; structure=SEQ ID NO:74), and 3H9-vc-MMAE. The ADC 3H9-DEVD-MMAE (name=SEQ ID NO:73; structure=SEQ ID NO:74) was prepared as described above for 3H9-KGDEVD-MMAE (name=SEQ ID NO: 65; structure—SEQ ID NO:66), except using maleimide-DEVD-MMAE (SEQ ID NO:3) in place of Maleimide-KGDEVD-MMAE (SEQ ID NO: 77).

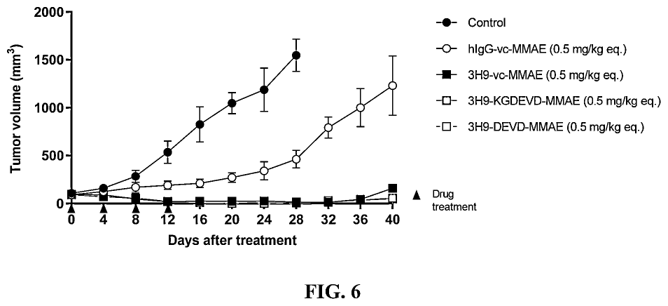

The results showed that the ADCs 3H9-KGDEVD-MMAE (name=SEQ ID NO: 65; structure=SEQ ID NO:66), 3H9-DEVD-MMAE (name=SEQ ID NO:73; structure=SEQ ID NO:74), and 3H9-vc-MMAE effectively suppressed tumor growth while a non-anti-doppel ADC (hIgG-vc-MMAE, having a human IgG moiety instead of a doppel-targeting antibody moiety) did not show a potent anti-tumor effect. (FIG. 6)

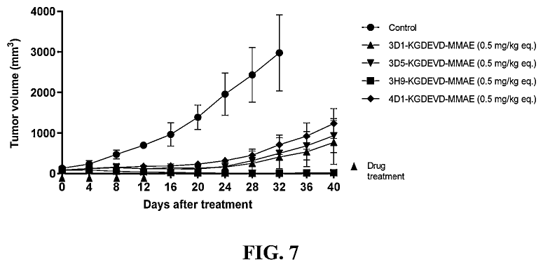

The efficacy of additional anti-doppel ADCs was assessed in the same tumor model described above, using the ADCs 3D1-KGDEVD-MMAE (name=SEQ ID NO:67; structure=SEQ ID NO:68), 3D5-KGDEVD-MMAE (name=SEQ ID NO: 69; structure=SEQ ID NO:70), 3H9-KGDEVD-MMAE (name=SEQ ID NO:65; structure=SEQ ID NO:66), and 4D1-KGDEVD-MMAE (name=SEQ ID NO: 71; structure=SEQ ID NO: 72) (prepared as described above, using the anti-doppel antibodies mAb 3D1, mAb 3D5, mAb 3H9, and mAb 4D1 in place of the antibodies used above). The results showed that each ADC tested effectively suppressed tumor growth. (FIG. 7).

The structures of the anti-doppel ADCs described in these examples are depicted in the table below.

| Name | |
|---|---|
| SEQ ID NOs | Anti-Doppel ADC Structures |
| 3H9- KGDEVD- MMAE SEQ ID NO: 65 (name) SEQ ID NO: 66 (structure) | 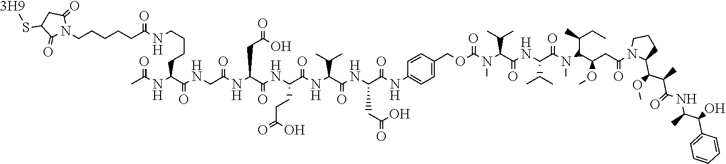 |
| 3D1- KGDEVD- MMAE SEQ ID NO: 67 (name) SEQ ID NO: 68 (structure) | 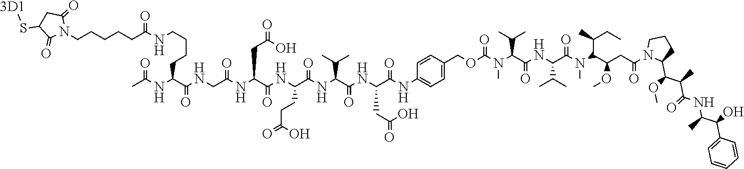 |
| 3D5- KGDEVD- MMAE SEQ ID NO: 69 (name) SEQ ID NO: 70 (structure) | 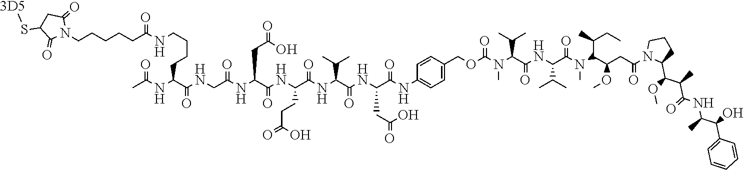 |
| 4D1- KGDEVD- MMAE SEQ ID NO: 71 (name) SEQ ID NO: 72 (structure) | 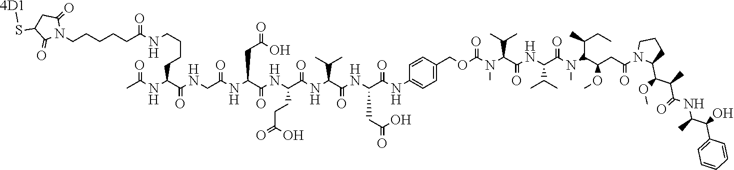 |
| 3H9-vc- MMAE | 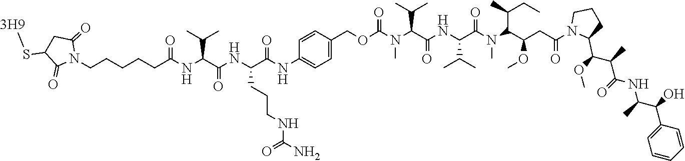 |
| 3D1-vc- MMAE | 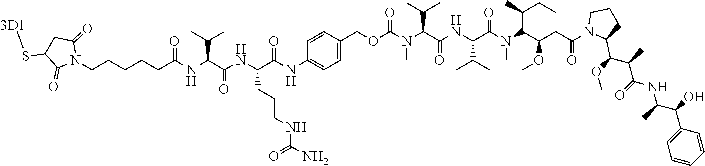 |
| 3D5-vc- MMAE | 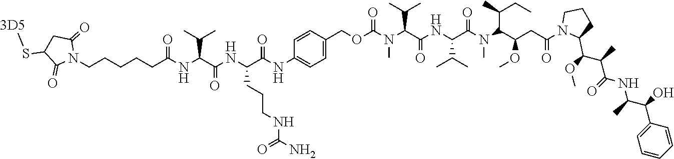 |
| 4D1-vc- MMAE | 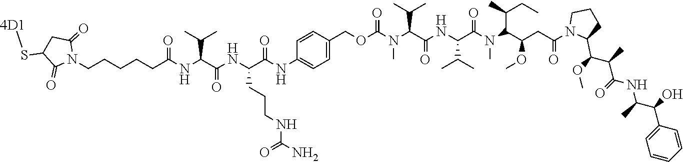 |
| 3H9- DEVD- MMAE SEQ ID NO: 73 (name) SEQ ID NO: 74 (structure) | 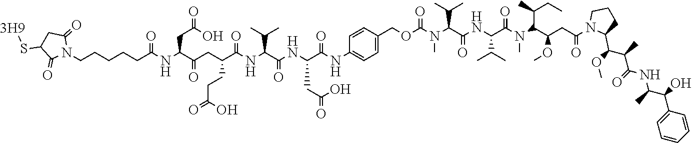 |
| 3H9- KGDEVD- Exatecan SEQ ID NO: 75 (name) SEQ ID NO: 76 (structure) | 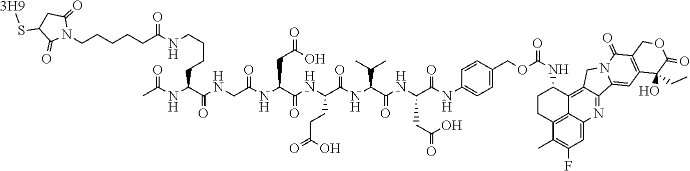 |

What is claimed is:

1. An anti-doppel antibody drug conjugate (ADC), comprising:
(i) a doppel-targeting moiety that binds to doppel, joined directly or through a linker to
(ii) a cleavable linker, joined directly or through a linker to
(iii) a therapeutic agent.

2. The ADC of claim 1, wherein the doppel-targeting moiety is selected from a monoclonal antibody, a polyclonal antibody, a single chain antibody, a chimeric antibody, a humanized antibody, a veneered antibody, and doppel-binding fragments of any thereof.

3. The ADC of claim 1, wherein the doppel-targeting moiety is a doppel-targeting antibody selected from human monoclonal antibody A12, human monoclonal antibody B2, human monoclonal antibody E9, human monoclonal antibody 3D5, human monoclonal antibody 3D1, human monoclonal antibody 4D1, human monoclonal antibody 3H9, and doppel-binding fragments of any thereof.

4. The ADC of claim 1, wherein the cleavable linker comprises a caspase-cleavable peptide linker.

5. The ADC of claim 4, wherein the caspase-cleavable peptide linker comprises four C-terminal amino acid residues selected from Asp-Xaa-Xaa-Asp, Leu-Xaa-Xaa-Asp, and Val-Xaa-Xaa-Asp, where Xaa represents any amino acid residue.

6. The ADC of claim 5, wherein the four C-terminal amino acid residues of the caspase-cleavable peptide linker are selected from Asp-Glu-Val-Asp (SEQ ID NO:4), Asp-Leu-Val-Asp (SEQ ID NO:5) Asp-Glu-Ile-Asp (SEQ ID NO:6), and Leu-Glu-His-Asp (SEQ ID NO:7).

7. The ADC of claim 1, wherein the caspase-cleavable peptide has an amino acid sequence consisting of Lys-Gly-Asp-Glu-Val-Asp (SEQ ID NO:8).

8. The ADC of claim 1, wherein the cleavable linker is cleavable by intracellular proteases.

9. The ADC of claim 1, wherein the cleavable linker is selected from a dipeptide cleavable linker wherein the dipeptide is selected from valine-citrulline, valine-alanine, and phenylalanine-lysine; a hydrazone linker hydrolyzed at a pH of less than 5.5; and a disulfide linker.

10. The ADC of claim 1, wherein the therapeutic agent comprises a chemotherapeutic agent that induces apoptosis of tumor cells.

11. The ADC of claim 10, wherein the chemotherapeutic agent is selected from 5-FU, afatinib, aplidin, azaribine, anastrozole, anthracyclines, axitinib, AVL-101, AVL-291, bendamustine, bleomycin, bortezomib, bosutinib, bryostatin-1, busulfan, calicheamycin, exatecan, camptothecin, carboplatin, 10-hydroxycamptothecin, carmustine, celebrex, chlorambucil, cisplatin, COX-2 inhibitors, irinotecan, SN-38, cladribine, crizotinib, cyclophosphamide, cytarabine, dacarbazine, dasatinib, dinaciclib, docetaxel, dactinomycin, daunorubicin, doxorubicin, epidophyllotoxin, erlotinib, entinostat, estrogen receptor binding agents, etoposide, exemestane, fingolimod, floxuridine, fludarabine, flutamide, flavopiridol, fostamatinib, ganetespib, GDC-0834, GS-1101, gefitinib, gemcitabine, hydroxyurea, ibrutinib, idarubicin, idelalisib, ifosfamide, imatinib, L-asparaginase, lapatinib, lenolidamide, leucovorin, LFM-A13, lomustine, mechloresthamine, melphalan, mercaptopurine, methotrexate, mitoxantrone, mithramycin, mitomycin, mitotane, navelbine, neratinib, nilotinib, nitrosurea, olaparib, plicomycin, procarbazine, paclitaxel, PCI-32765, pentostatin, PSI-341, raloxifene, semustine, sorafenib, streptozocin, SU11248, sunitinib, tamoxifen, temazolomide, transplatin, thalidomide, thioguanine, thiotepa, teniposide, topotecan, uracil mustard, vatalanib, vinorelbine, vinblastine, vincristine, vinca alkaloids, and ZD183.

12. The ADC of claim 10, wherein the chemotherapeutic agent is selected from anthracyclines, antibiotics, alkylating agents, platinum-based agents, antimetabolites, topoisomerase inhibitors, and mitotic inhibitors.

13. The ADC of claim 10, wherein the chemotherapeutic agent is selected from doxorubicin, daunorubicin, epirubicin, idarubicin, valrubicin, and derivatives thereof.

14. The ADC of claim 10, wherein the chemotherapeutic agent is selected from the group consisting of actinomycin-D, bleomycin, mitomycin-C, calicheamicin, and derivatives thereof.

15. The ADC of claim 10, wherein the chemotherapeutic agent is selected from cyclophosphamide, mechlorethamine, uramustine, melphalan, chlorambucil, ifosfamide, bendamustine, carmustine, lomustine, streptozocin, busulfan, dacarbazine, temozolomide, thiotepa, altretamine, duocarmycin, cisplatin, carboplatin, nedaplatin, oxaliplatin, satraplatin, triplatin tetranitrate, 5-fluorouracil, 6-mercaptopurine, capecitabine, cladribine, clofarabine, cytarabine, floxuridine, fludarabine, gemcitabine, hydroxyurea, methotrexate, pemetrexed, pentostatin, thioguanine, exatecan, camptothecin, topotecan, irinotecan, etoposide, teniposide, mitoxantrone, paclitaxel, docetaxel, ixabepilone, vinblastine, vincristine, vindesine, vinorelbine, estramustine, maytansine, DM1 (mertansine), DM4, dolastatin, auristatin E, auristatin F, monomethyl auristatin E (MMAE), and derivatives thereof.

16. The ADC of claim 10, wherein the chemotherapeutic agent is monomethyl auristatin E (MMAE).

17. The ADC of claim 10, wherein the chemotherapeutic agent is exatecan.

18. The ADC of claim 1, wherein the therapeutic agent comprises an immunomodulatory agent.

19. The ADC of claim 18, wherein the immunomodulatory agent is selected from cytokines, lymphokines, monokines, stem cell growth factors, lymphotoxins, hematopoietic factors, colony stimulating factors (CSF), interrerons (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, transforming growth factor (TGF), TGF-alpha, TGF-beta, insulin-like growth factor (IGF), erythropoietin, thrombopoietin, tumor necrosis factor (TNF), TNF-alpha, TNF-beta, mullerian-inhibiting substance, mouse gonadotropin-associated peptide, inhibin, activin, vascular endothelial growth factor, integrin, interleukin (IL), granulocyte-colony stimulating factor (G-CSF), granulocyte macrophage-colony stimulating factor (GM-CSF), interferon-alpha, interferon-beta, interferon-gamma, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-23, IL-25, LIF, kit-ligand, FLT-3, angiostatin, thrombospondin, endostatin, toll-like receptor (TLR) agonists (optionally selected from CU-T12-9, Pam3CSK4, FSL-1, Pam2CSK4, and CL429), Poly(A:U), Poly(I:C), lipopolysaccharides (LPS), MPLA-SM, CRX-527, flagellin, thiazoquinoline derivatives, imidazoquinoline derivatives (optionally selected from CL097, gardiquimod, imiquimod, and resiquimod), adenine analogs, guanosine analogs, thymidine analogs, benzoazepine analogs, and CpG oligodeoxynucleotides (ODN) (optionally selected from ODN 1585, ODN 2216, ODN 2336, ODN 1668, ODN 1826, ODN 2006, ODN 2007, ODN BW006, ODN D-SL01, ODN 2395, ODN M362, and ODN D-SL03).

20. The ADC of claim 1, wherein the therapeutic agent comprises a toxin.

21. The ADC of claim 20, wherein the toxin is selected from ricin, abrin, ribonuclease (RNase), DNase 1, Staphylococcal enterotoxin-A, pokeweed antiviral protein, gelonin, diphtheria toxin, Pseudomonas exotoxin, and Pseudomonas endotoxin.

22. The ADC of claim 1, wherein the therapeutic agent comprises a radionuclide.

23. The ADC of claim 22, wherein the radionuclide is selected from 11C, 13N, 15O, 32P, 33P, 47Sc, 51Cr, 57Co, 58Co, 59Fe, 62Cu, 67Cu, 67Ga, 75Br, 75Se, 76Br, 77As, 77Br, 80mBr, 89Sr, 90Y, 95Ru, 97Ru, 99Mo, 99mTc, 103mRh, 103Ru, 105Rh, 105Ru, 107Hg, 109Pd, 109Pt, 111Ag, 111In, 113mIn, 119Sb, 121mTe, 122mTe, 125I, 125mTe, 126I, 131I, 133I, 142Pr, 143Pr, 149Pm, 152Dy, 153Sm, 161Ho, 161Tb, 165Tm, 166Dy, 166Ho, 167Tm, 168TM, 169Er, 169Yb, 177Lu, 186Re, 188Re, 189mOs, 189Re, 198Ir, 194Ir, 197Pt, 198Au, 199Au, 203Hg, 211At, 211Bi, 211Pb, 212Bi, 212Pb, 213Bi, 215Po, 217At, 219Rn, 221Fr, 223Ra, 225Ac, 227Th and 255Fm.

24. The ADC of claim 1, wherein the therapeutic agent is a DNA cross-linking agent selected from indolionobenzodiazepine dimer (IGN), pyrrolobenzodiazepine (PBD) and derivatives thereof.

25. The ADC of claim 1, wherein the ADC is selected from 3D1-KGDEVD-MMAE (name=SEQ ID NO:67; structure=SEQ ID NO:68), 3D5-KGDEVD-MMAE (name=SEQ ID NO:69; structure=SEQ ID NO:70), 3H9-KGDEVD-MMAE (name=SEQ ID NO:65; structure=SEQ ID NO:66), and 4D1-KGDEVD-MMAE (name=SEQ ID NO:71; structure=SEQ ID NO: 72); 3H9-vc-MMAE; 3D1-vc-MMAE; 3D5-vc-MMAE; 4D1-vc-MMAE; 3H9-DEVD-MMAE (name=SEQ ID NO:73; structure=SEQ ID NO:74), and 3H9-KGDEVD-Exatecan (name=SEQ ID NO:75; structure=SEQ ID NO:76).

26. A pharmaceutical composition comprising the ADC of claim 1 and a pharmaceutically acceptable carrier.

27. The pharmaceutical composition of claim 26, formulated for intravenous administration.

28. A method for treating a doppel-associated disease or condition in a subject, comprising administering to a subject in need thereof an ADC according to claim 1.

29. The method of claim 28, wherein the doppel-associated disease or condition is selected from asthma, tuberculosis, atherosclerosis, and pulmonary arterial hypertension (PAH).

30. The method of claim 28, wherein the doppel-associated disease or condition is a cancer, wherein cells of the cancer express doppel.

31. A kit comprising the ADC according to claim 1 contained in a container, optionally further comprising instructions for use.
